(19) United States
(12) Patent Application Publication
(10) Pub. No.: US 2025/0085275 A1
(43) Pub. Date: Mar. 13, 2025

(54) FLUORESCENCE TAGGING OF GLYCANS AND OTHER BIOMOLECULES THROUGH REDUCTIVE AMINATION FOR ENHANCED MS SIGNALS

(71) Applicant: Waters Technologies Corporation, Milford, MA (US)

(72) Inventors: Darryl W. Brousmiche, Grafton, MA (US); Matthew A. Lauber, North Smithfield, RI (US)

(73) Assignee: Waters Technologies Corporation, Milford, MA (US)

(21) Appl. No.: 18/941,823

(22) Filed: Nov. 8, 2024

Related U.S. Application Data
(63) Continuation of application No. 17/338,022, filed on Jun. 3, 2021.
Application No. 17/338,022 is a continuation of application No. 16/312,826, filed on Dec. 21, 2018, now Pat. No. 11,061,023.
(60) Provisional application No. 62/352,724, filed on Jun. 21, 2016.

(51) Int. Cl. G01N 33/533; C07D 215/48

(52) U.S. Cl. CPC G01N 33/533; C07D 215/48

(57) ABSTRACT

Novel reagents comprising MS active, fluorescent compounds having an activated functionality for reaction with aldehydes and useful in labeling biomolecules such as glycans and methods of making the same are taught and described.

### CROSS REFERENCE TO RELATED APPLICATIONS

This application is a continuation application of U.S. patent application Ser. No. 17/338,022, filed Jun. 3, 2021, which is a continuation application of U.S. patent application Ser. No. 16/312,826, filed Dec. 21, 2018, which is the U.S. National Phase of International Application No. PCT/US2017/038070, filed Jun. 19, 2017, which claims priority to U.S. Provisional Patent Application No. 62/352,724, filed Jun. 21, 2016. The entire contents of these applications are incorporated herein by reference.

### BACKGROUND OF THE INVENTION

Analysis of glycans is used for protein research and can be important to clinical chemists and pharmaceutical manufacturers, especially where glycosylation profiling of proteins is monitored to ensure consistency of a therapeutic product. As such, fluorescent labeling of glycans is beneficial because the sensitivity and selectivity of glycan detection can be improved as well as the chromatographic behavior. However, upon derivation with a reagent having the fluorescent moiety, the functional group of the compound is estimated. Mass spectrometry (“MS”) is then required to identify the specific compound. Furthermore, certain tagging reagents have good fluorescence signal, but a poor MS signal.

There is a need, therefore, for compounds which can react with glycans and other biomolecules to provide a derivative compound (or one that is tagged or labeled by the reagent), and/or a conjugate glycan, that produces reliable mass spectrometry and fluorescence signals.

### SUMMARY OF THE INVENTION

Provided herein are MS active compounds useful in fluorescence labeling of glycans such as oligosaccharides, N-linked glycans, O-linked glycans and other biomolecules including, but not limited to, proteins and peptides that contain an aldehyde group or a ketone group. These MS active, fluorescent compounds have three functional components: (a) a tertiary or quaternary amino group or other MS active atom; (b) a highly fluorescent moiety, and (c) an amine group that can react with a ketone or aldehyde group of the glycan or other biomolecule. The amine group provides effective labeling of glycans through reductive amination. The fluorescent moiety provides the fluorescent signal. The tertiary amino group (otherwise sometimes referred to herein as the MS active atom) provides a strong MS signal.

In particular, compounds of the various formulas are described herein. Each compound can be a reagent for fluorescence labeling and enhanced MS signaling of glycans and other biomolecules. The terms “labeling” and “tagging” are used interchangeably through this specification.

The MS active, fluorescence tagging compounds can be of the structural Formula I:

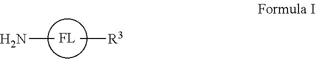

and salts or solutes thereof,
wherein
FL is a fluorophore, such as a phenyl, quinoline, naphthalene, coumarin, quinolinones or rhodamine compound;
R³ is

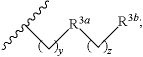

R^{3a} is selected from ester, amide, amine, oxygen, urea, carbamate, carbonate, sulfur, thiourea, thiocarbamate, alkyl or carbonyl;
R^{3b} is

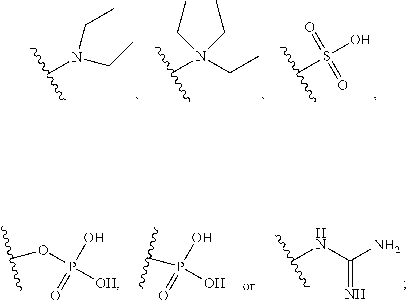

y=0−12; and
z=1−12.

In an embodiment, the compound of Formula I is selected from

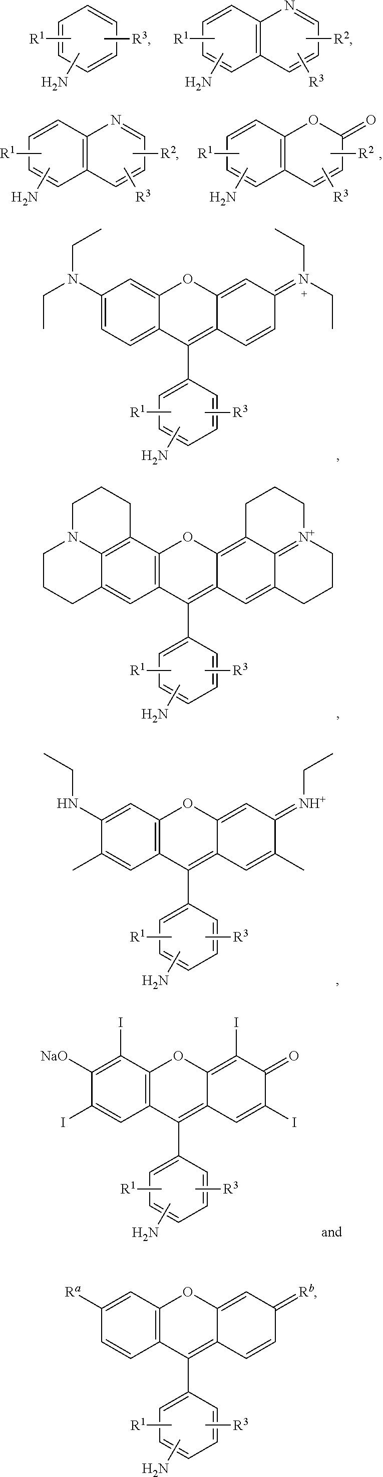

and salts or solvates thereof, wherein
R¹ and R² are independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R₃ is

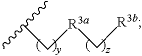

R₃ₐ is selected from ester, amide, amine, oxygen, urea, carbamate, carbonate, sulfur, thiourea, thiocarbamate, alkyl or carbonyl;
R^{3b} is

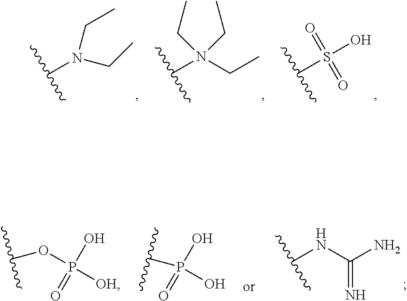

y=0−12;
z=1−2;
R^{a} is selected from

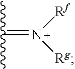

R^{b} is oxo, or

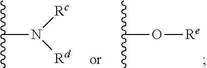

and
R^{c}, R^{d}, R^{e}, R^{f} and R^{g} are independently selected from hydrogen and optionally substituted alkyl.

The compounds described herein can have optical centers and therefore can occur in different enantiomeric and disastereomeric configurations. The present compounds further include enantiomers, diastereomers and other stereoisomers of such compounds of each formula, as well as racemic compounds and racemic mixtures and other mixtures of stereoisomers thereof.

### DETAILED DESCRIPTION

Biopolymers, such as glycans, play significant roles in physiological and pathological processes. Labeling (otherwise referred to herein as “tagging”) glycans with fluorescent reagent compounds can improve detection of the glycan. Quantitative analysis of glycans from normal and disease specimens can provide insight into disease onset and progression. Relative glycan quantification can be accomplished through modification of the glycans with either chromogenic or fluorogenic tags for optical measurement or isotopic tags for mass spectrometric analysis. Yang et. al., Glycan Analysis by Isobaric Aldehyde Reactive Tags and Mass Spectrometry, 85 ANAL CHEM. 8188 (2013). The ion abundance of N-linked glycans in electrospray ionization mass spectrometry (“ESI MS”) can be increased by derivatizing the glycans with neutral, hydrophobic reagents. Walker et. al., Hydrophobic Derivatization of N-linked Glycans for Increased Ion Abundance in Electrospray Ionization Mass Spectrometry, 22 J. AM. SOC. MASS SPECTROM. 1309 (2011). In other words, hydrophobic derivatization of glycans can increase ion abundance in ESI MS. In addition, we have uncovered that by using a reagent or tagging compound having a high pKa, MS signaling of the derivatized or tagged glycan is further enhanced.

Hence, provided herein are novel compounds useful in the fluorescence tagging of glycans and with enhanced MS signaling such as N-linked glycans O-linked glycans and other bio-molecules including, but not limited to, proteins, peptides and amino acids. These compounds are useful to analyze glycans and/or other biomolecules in a sample. To analyze a glycan or other biomolecule, the glycan can be labeled with one of the compounds described herein and then subjected to liquid chromatography, and mass spectrometry and fluorescence detection.

The term “alkoxy,” as used herein, alone or in combination, refers to an alkyl ether radical, wherein the term alkyl is as defined below. Examples of suitable alkyl ether radicals include methoxy, ethoxy, n-propoxy, isopropoxy, n-butoxy, iso-butoxy, sec-butoxy, tert-butoxy, and the like.

The term “alkyl,” as used herein, alone or in combination, refers to a straight-chain or branched-chain alkyl radical containing from 1 to and including 20, preferably 1 to 10, and more preferably 1 to 6, carbon atoms. Alkyl groups can be optionally substituted as defined herein without changing or effecting the fluorescent or mass spec properties of the molecule. Examples of alkyl radicals include methyl, ethyl, n-propyl, isopropyl, n-butyl, isobutyl, sec-butyl, tert-butyl, pentyl, iso-amyl, hexyl, octyl, nonyl and the like. The term “alkylene,” as used herein, alone or in combination, refers to a saturated aliphatic group derived from a straight or branched chain saturated hydrocarbon attached at two or more positions, such as methylene (—CH₂—).

The term “alkylamino,” as used herein can be a mono- or dialkylated groups (also referred to “dialkylamino”) such as, for example, N-methylamino, N-ethylamino, N,N-dimethylamino, N,N-ethylmethylamino and the like and combination, refers to —NRR′, wherein R is independently selected from the group consisting of hydrogen and alkyl, and R′ is alkyl, any of which can themselves be optionally substituted and the dialkyamino group can further comprise a spacer (sometimes referred to as a linker or linker group). A molecular spacer or simply a “spacer” in chemistry is any part of a molecule that provides a connection between two other functional parts of a molecule, for example, the rapid reacting portion, the MS active portion and the fluorescent portion.

The term “parent molecular moiety” as used herein means and includes a phenyl, quinoline, naphthalene, coumarin, quinolinones or rhodamine.

The term “amino,” as used herein, alone or in combination, refers to —NRR′, wherein R and R′ are independently selected from the group consisting of hydrogen, alkyl, acyl, heteroalkyl, aryl, cycloalkyl, heteroaryl, and heterocycloalkyl, any of which can themselves be optionally substituted.

The term “aryl,” as used herein, alone or in combination, means a carbocyclic aromatic system containing one, two or three rings wherein such rings can be attached together in a pendent manner or can be fused. The term “aryl” embraces aromatic radicals such as benzyl, phenyl, naphthyl, anthracenyl, phenanthryl, indanyl, indenyl, annulenyl, azulenyl, tetrahydronaphthyl, heteroaryl (e.g., pyridine) and biphenyl.

The terms “benzo” and “benz,” as used herein, alone or in combination, refer to the divalent radical C₆H₄═ derived from benzene. Examples include benzothiophene and benzimidazole.

The term “carbamate,” as used herein, alone or in combination, refers to an ester of carbamic acid (—NHCOO—) which can be attached to the parent molecular moiety from either the nitrogen or acid end, and which can be optionally substituted as defined herein.

The term “O-carbamyl” as used herein, alone or in combination, refers to a —OC(O)NRR′, group-with R and R′ as defined herein.

The term “N-carbamyl” as used herein, alone or in combination, refers to a ROC(O) NR′— group, with R and R′ as defined herein.

The term “carbonyl,” as used herein, when alone includes formyl [—C(O)H] and in combination is a —C(O)— group.

The term “carboxy,” as used herein, refers to —C(O)OH or the corresponding “carboxylate” anion, such as is in a carboxylic acid salt. An “O-carboxy” group refers to a RC(O)O— group, where R is as defined herein. A “C-carboxy” group refers to a —C(O)OR groups where R is as defined herein.

The term “cycloalkyl” refers to a carbocyclic substituent obtained by removing a hydrogen from a saturated carbocyclic molecule and having three to fourteen carbon atoms. In one embodiment, a cycloalkyl substituent has three to ten carbon atoms. Examples of cycloalkyl include cyclopropyl, cyclobutyl, cyclopentyl and cyclohexyl.

The term “halo,” or “halogen,” as used herein, alone or in combination, refers to fluorine, chlorine, bromine, or iodine.

The term “haloalkoxy,” as used herein, alone or in combination, refers to a haloalkyl group attached to the parent molecular moiety through an oxygen atom.

The term “haloalkyl,” as used herein, alone or in combination, refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, can have an iodo, bromo, chloro or fluoro atom within the radical. Dihalo and polyhaloalkyl radicals can have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like.

The term “heteroalkyl,” as used herein, alone or in combination, refers to a stable straight or branched chain, or cyclic hydrocarbon radical, or combinations thereof, fully saturated or containing from 1 to 3 degrees of unsaturation, consisting of the stated number of carbon atoms and from one to three heteroatoms selected from the group consisting of O, N, and S, and wherein the nitrogen and sulfur atoms can optionally be oxidized and the nitrogen heteroatom can optionally be quaternized. The heteroatom(s) O, N and S can be placed at any interior position of the heteroalkyl group. Up to two heteroatoms can be consecutive, such as, for example, —CH₂—NH—OCH_{3.}

The terms “heterocycloalkyl” and, interchangeably, “heterocycle,” as used herein, alone or in combination, each refer to a saturated, partially unsaturated, or fully unsaturated monocyclic, bicyclic, or tricyclic heterocyclic radical containing at least one, preferably 1 to 4, and more preferably 1 to 2 heteroatoms as ring members, wherein each said heteroatom can be independently selected from the group consisting of nitrogen, oxygen, and sulfur, and wherein there are preferably 3 to 8 ring members in each ring, more preferably 3 to 7 ring members in each ring, and most preferably 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Heterocycle groups of the compounds are exemplified by aziridinyl, azetidinyl, 1,3-benzodioxolyl, dihydroisoindolyl, dihydroisoquinolinyl, dihydrocinnolinyl, dihydrobenzodioxinyl, dihydro[1,3]oxazolo[4,5-b]pyridinyl, benzothiazolyl, dihydroindolyl, dihy-dropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups can be optionally substituted unless specifically prohibited.

The term “optionally substituted” means the anteceding group can be substituted or unsubstituted. When substituted, the substituents of an “optionally substituted” group can include, without limitation, one or more substituents independently selected from the following groups or a specific designated set of groups, alone or in combination: lower alkyl, lower alkenyl, lower alkynyl, lower alkanoyl, lower heteroalkyl, lower heterocycloalkyl, lower haloalkyl, lower haloalkenyl, lower haloalkynyl, lower perhaloalkyl, lower perhaloalkoxy, lower cycloalkyl, phenyl, aryl, aryloxy, lower alkoxy, lower haloalkoxy, oxo, lower acyloxy, carbonyl, carboxyl, lower alkylcarbonyl, lower carboxyester, lower carboxamido, cyano, hydrogen, halogen, hydroxy, amino, lower alkylamino, arylamino, amido, nitro, thiol, lower alkylthio, arylthio, lower alkylsulfinyl, lower alkylsulfonyl, arylsulfinyl, arylsulfonyl, arylthio, sulfonate, sulfonic acid, trisubstituted silyl, N₃, SH, SCH₃, C(O)CH₃, CO₂CH₃, CO₂H, pyridinyl, thiophene, furanyl, lower carbamate, and lower urea. Two substituents can be joined together to form a fused five-, six-, or seven-membered carbocyclic or heterocyclic ring consisting of zero to three heteroatoms, for example forming methylenedioxy or ethylenedioxy. An optionally substituted group can be unsubstituted (e.g., —CH₂CH₃), fully substituted (e.g., —CF₂CF₃), monosubstituted (e.g., —CH₂CH₂F) or substituted at a level anywhere in-between fully substituted and monosubstituted (e.g., —CH₂CF₃). Where substituents are recited without qualification as to substitution, both substituted and unsubstituted forms are encompassed. Where a substituent is qualified as “substituted,” the substituted form is specifically intended. Additionally, different sets of optional substituents to a moiety can be defined as needed; in these cases, the optional substitution will be as defined, often immediately following the phrase, “optionally substituted with.”

The term R or the term R′, appearing by itself and without a number designation, unless otherwise defined, refers to a moiety selected from the group consisting of hydrogen, alkyl, cycloalkyl, heteroalkyl, aryl, heteroaryl and heterocycloalkyl, any of which can be optionally substituted. Such R and R′ groups should be understood to be optionally substituted as defined herein. Whether an R group has a number designation or not, every R group, including R, R′ and Rⁿ where n=(1, 2, 3, . . . n), every substituent, and every term should be understood to be independent of every other in terms of selection from a group. Should any variable, substituent, or term (e.g. aryl, heterocycle, R, etc.) occur more than one time in a formula or generic structure, its definition at each occurrence is independent of the definition at every other occurrence. Those of skill in the art will further recognize that certain groups can be attached to a parent molecular moiety or can occupy a position in a chain of elements from either end as written. Thus, by way of example only, an unsymmetrical group such as —C(O)N(R)— can be attached to the parent molecular moiety at either the carbon or the nitrogen.

The term “bond” refers to a covalent linkage between two atoms, or two moieties when the atoms joined by the bond are considered to be part of larger substructure. A bond can be single, double, or triple unless otherwise specified. A dashed line between two atoms in a drawing of a molecule indicates that an additional bond can be present or absent at that position. The development and production of therapeutic proteins is becoming the fastest-growing segment of the pharmaceutical industry. The efficacy, stability and protein secretion of these large molecule drugs depend on their Post Translational Modifications (“PTMs”). Glycosylation is the most complex and common PTM and plays a vital role in the safety and efficacy of many therapeutic proteins such as recombinant antibodies. Several studies have shown the correlation between glycosylation variations caused by cell line selection and changes in culture medium parameters. Patrick Hossler et al., Optimal and Consistent Protein Glycosylation in Mammalian Cell Culture, 19 GLYCOBIOLOGY 926 (2009). These variations can have a profound effect on the biological activities of the mAb drugs, which leads to changes in drug potency in the final product. Regulatory agencies require monitoring of batch-to-batch recombinant antibody drug production quality and mandate detailed assessment of the protein glycosylation micro-heterogeneity and consistency.

The term “urea” means and includes a compound having the chemical formula CO(NH₂)₂ where the molecule has two —NH₂ groups joined by a carbonyl (C═O) functional group

The compounds described herein can also form hydrogen bonds with other compounds. A hydrogen bond is an electromagnetic attractive interaction between polar molecules, where hydrogen is bonded to an electronegative atom such as nitrogen or oxygen. The hydrogen bond represents a strong dipole-dipole attraction. These hydrogen-bond attractions can occur between molecules (intermolecular) or within different parts of a single molecule (intramolecular). When a hydrogen atom is attached to an electronegative atom, it is considered a hydrogen bond donor. The electronegative atom is considered a hydrogen bond acceptor, whether it is bonded to a hydrogen atom or not.

Asymmetric centers exist in the compounds presented herein. These centers are designated by the symbols “R” or “S,” depending on the configuration of substituents around the chiral carbon atom. It should be understood that the compounds encompasses all stereochemical isomeric forms, including diastereomeric, enantiomeric, and epimeric forms, as well as d-isomers and 1-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of certain stereochemistry are either commercially available or can be made and resolved by techniques known in the art. Additionally, the compounds described can exist as geometric isomers and includes all cis, trans, syn, anti, entgegen (E), and zusammen (Z) isomers as well as the appropriate mixtures thereof. Additionally, compounds can exist as tautomers. All tautomeric isomers are provided. Additionally, the present compounds can exist in unsolvated as well as solvated forms with pharmaceutically acceptable solvents such as water, ethanol, and the like. In general, the solvated forms are considered equivalent to the unsolvated forms.

Hence, the compounds described herein can also be in the form of a salt or solvate, or acid addition salts. Through a reaction with either organic or inorganic acids, compounds presented herein or groups of compounds can form a salt. For example, in acid-base neutralization, an acid and a base react to form water and a salt. Basically, to react together, there must be the transfer of protons between acids and bases. Also, different acids can produce different ions. For example, an Arrhenius acid produces hydronium ions when it dissociates in water. Similarly, a Bronsted-Lowry acid is a proton donor that donates hydrogen ions to the base. Hence, proton acceptors and proton donors are the basis for the reaction and are referred to sometimes as a conjugate base or a conjugate acid. A conjugate pair refers to acids and bases with common features, where there is an equal loss/gain of protons between the pairs. For example NH₄⁺ is the conjugate acid to the base NH₃ because NH₃ gains a hydrogen ion to form NH₄⁺ as H₂O donates a hydrogen ion to form OH⁻, the conjugate base. On the other hand, under a different theory, a Lewis acid accepts an electron pair and a Lewis base donates an electron pair donor. Accordingly, the proton H⁺ can be an electron pair acceptor. Moreover, a compound can be both, a Lewis acid and a Lewis base, depending on the reaction. For example, methyl iodide can behave as both, a Lewis acid and a Lewis base, where the methyl group is donated to form a salt.

The compounds of the formulas described herein can have one or more quaternary nitrogen. The quaternary nitrogen has a positive charge on the nitrogen and can be associated with a counterion and include all quaternary amine-counterion complexes of compounds when a compound includes a quaternary amine group.

The terms tagging, conjugating and derivatizing when referred to in the context of an association between a compound of Formula I through Formula X refers to the bond formation of one of the compounds with an aldehyde containing compound.

The term “oxo” indicates that the chemical compound contains oxygen linked to another atom by a double bond and can denote that the compound is derived from a specified compound by replacement of a methylene group with a carbonyl group. In addition, oxo is sometimes used as a prefix (i.e., in IUPAC nomenclature) for the functional group ═O, a substituent oxygen atom connected to another atom by a double bond.

Examples of acids which can be employed to form a salt of any of the compounds provided herein include inorganic acids and organic acids as well known to those skilled in the art such as, but not limited to, N-hydroxysuccinimide, hydrochloric, hydrofluoric, hydroiodic, hydrobromic, sulfuric, hydrosulfuric, thiosulfuric, hydrocyanic, phosphoric, phosphorous, hydrochlorous, chlorous, nitrous, nitric, chloric, perchloric, sulfurous, oxalic, maleic, succinic, and citric. Salts can also be formed by coordination of the compounds with an alkali metal or alkaline earth ion. In addition, other acids can form a salt including, but not limited to, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, acetic acid, adipic acid, ascorbic acid (L), aspartic acid (L), benzenesulfonic acid, benzoic acid, camphoric acid (+), camphor-10-sulfonic a cid (+), capric acid (decanoic acid), caproic acid (hexanoic acid), caprylic acid (octanoic acid), carbonic acid, cinnamic acid, citric acid, cyclamic acid, dodecylsulfuric acid, ethane-1,2-disulfonic acid, ethanesulfonic acid, ethanesulfonic acid, formic acid, fumaric acid, galactaric acid, gentisic acid, glucoheptonic acid (D), gluconic acid (D), glucuronic acid (D), glutamic acid, glutaric acid, glycerophosphoric acid, isobutyric acid, lactic acid (DL), lactobionic acid, lauric acid, maleic acid, malic acid (−L), malonic acid, mandelic acid (DL), methanesulfonic acid, naphthalene-1,5, disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (−L), salicylic acid, sebacic acid, stearic acid, succinic acid, sulfuric acid, tartaric acid (+L), thiocyanic acid, toluenesulfonic acid (p), undecylenic acid.

For the compounds described herein, the counterion can be the conjugate base formed after reacting a compound or groups of compounds with an acid. In other words, counterion holds the opposite charge to that of the compound or compounds it is associated with. Thus, with respect to possible salts of the compounds herein having a conjugate acid of NH₄⁺, the counterion represents the anionic part of the salt. In addition, it can be possible to have four organic substituents on the nitrogen. These species are not amines but are quaternary ammonium cations having a charged nitrogen center. Quaternary ammonium salts can exist with many kinds of anions.

Hence, counterions of a salt compound described herein can include, but are not limited to, any of the following common anions and oxoanions: N-hydroxysuccinimidyl, hydride (H⁻), fluoride (F⁻), chloride (Cl⁻), bromide (Br⁻), iodide (I⁻), oxide (O²⁻), hydroxide (OH⁻), peroxide (O₂²⁻), sulfide (S²⁻), hydrogen sulfide (HS⁻), selenide (Se²⁻), nitride (N³⁻), azide (N₃⁻), phosphide (P³⁻), arsinide (As³⁻), carbide (C⁴⁻), cyanide (CN⁻), hypochlorite (ClO₁⁻), chlorite (ClO₂⁻), chlorate (ClO₃⁻), perchlorate (ClO₄⁻), sulfite (SO₃²⁻), sulfate (SO₄²⁻), hydrogen sulfate (HSO₄⁻), thiosulfate (S₂O₃²⁻), nitrite (NO₂⁻), nitrate (NO₃⁻), phosphite (PO₃²⁻), phosphate (PO₄³⁻), (mono)hydrogen phosphate (HPO₄²⁻), dihydrogen phosphate (H2PO₄⁻), carbonate (CO₃²⁻), hydrogen carbonate (HCO₃⁻), oxalate (C₂O₄²⁻), cyanate (NCO⁻), isocyanate (OCN⁻), thiocyanate (SCN⁻), chromate (CrO₄²⁻), dichromate (Cr₂O₇²⁻), permanganate (MnO₄⁻).

### Derivatization Through Reductive Amination

Compounds for labeling biomolecules such as N and O glycans, with MS active fluorescent compounds of Formula I, as well as conjugates resulting therefrom are provided. In an embodiment, a biomolecule (such as a glycan) are tagged, derivatized or conjugated through an aldehyde or ketone with an amine of one or more of the compounds provided herein containing fluorescent, MS active properties through reductive amination. If a carbonyl functionality (e.g. ketone, aldehyde) is present on the reagent, it is possible that the reagent could self-react and/or form linear polymers under the conditions of reductive amination. Therefore, considerations must be made towards the same.

General conditions for the reductive amination reaction can be applied for tagging. For example, the reaction can be conducted in the presence reducing agents such as sodium cyanoborohydride or sodium triacetoxyborohydride. The reaction can be conducted in a mixture of citric acid and/or acetic acid with an organic solvent such as dimethylsulfoxide. The reaction can also be conducted in a solvent selected from tetrahydrofuran, dichloromethane, 1,2-dichloroethane, ethanol, methanol or isopropanol, toluene and xylene, and mixtures thereof.

The following schematic depicting labeling (tagging) a glycan using a compound of Formula I through reductive amination:

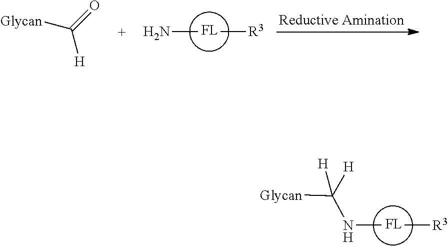

wherein FL and R₃ are as described above.

The following schematic shows the tagging of an amine containing glycan through reductive amination:

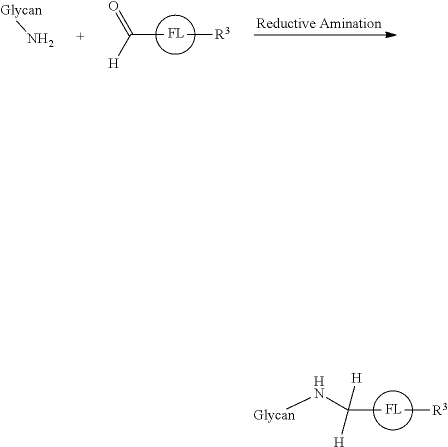

wherein FL and R₃ are as described above.

### Quinoline Based MS Active Fluorescence Tagging Compounds

MS active, fluorescence tagging compounds can be a quinoline derivative of the structural Formula II:

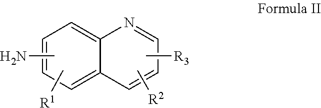

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R₃ is

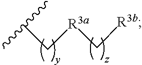

R₃ₐ is selected from ester, amide, amine, oxygen, urea, carbamate, carbonate, sulfur, thiourea, thiocarbamate, alkyl or carbonyl;
R^{3b} is

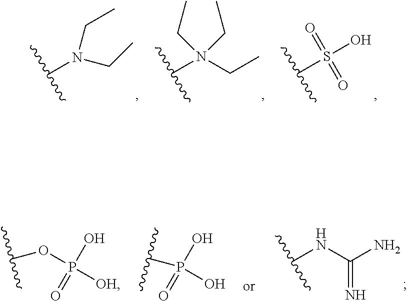

y=0−12;
z=1−12;
and salts or solvates thereof.

Compounds of structural Formula II are provided, with the proviso that said compound of Formula II is other than 6-amino-N-[2-(diethylamino)ethyl]-2-quinolinecarboxamide, and with the proviso that when y is one, R^{3a} is amide, R^{3b} is other than

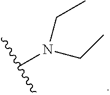 [SMILES: *N(CC)CC]

In an embodiment, the compound of Formula II, with the proviso that when y is zero, R^{3a} is amine, oxygen or sulfur and z is two, R^{3b} is other than

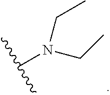 [SMILES: *N(CC)CC]

In an embodiment, the compound of Formula II, with the proviso that when y is one and R^{3a} is an amide, and z is two or three, R^{3b} is other than

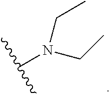 [SMILES: *N(CC)CC]

In yet another embodiment, compounds of Formula IIA are provided as follows:

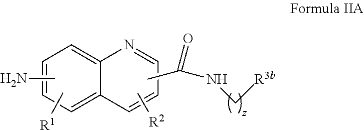

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

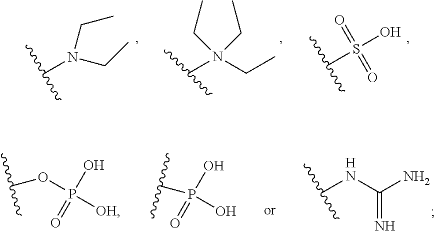

z=1−12;
and salts or solvates thereof.

In an embodiment, the compound of Formula IIA is other than 6-amino-N-[2-(diethylamino)ethyl]-2-quinolinecarboxamide.

Compounds of the structural Formula IIB are provided as follows:

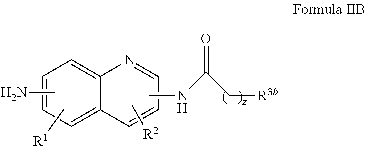

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

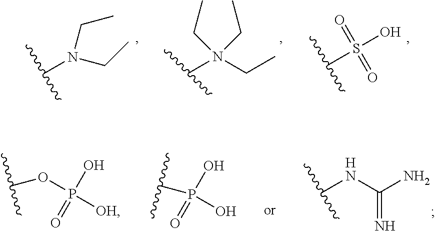

z=1−12;
and salts or solvates thereof.

Compounds of the structural Formula IIC are provided as follows:

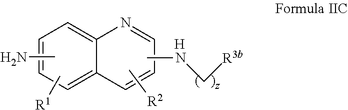

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

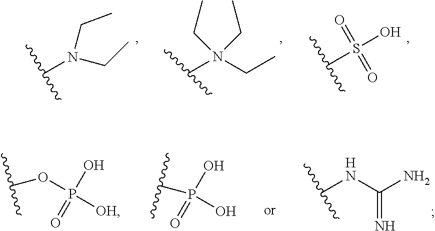

z=1−12;
and salts or solvates thereof.

In an embodiment, the compound of Formula IIC is provided with the proviso that when z is two, R^{3b} is other than

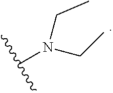 [SMILES: *N(CC)CC]

Compound of Formula IID are provided as followed:

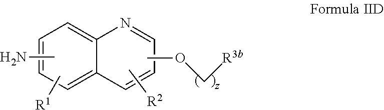

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

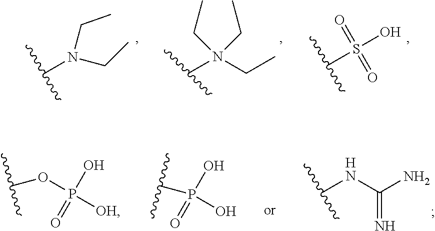

z=1−12;
and salts or solvates thereof.

In an embodiment, a compound of Formula IID is provided with the proviso that when z is two, R^{3b} is other than

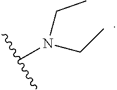 [SMILES: *N(CC)CC]

Also provided are compounds of the structural Formula IIE:

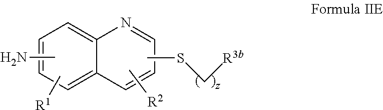

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

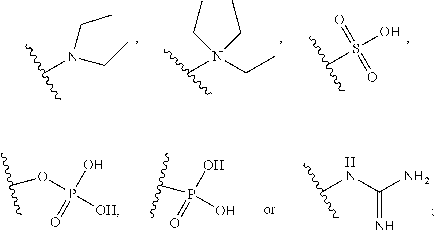

z=1−12;
and salts or solvates thereof.

In an embodiment, compounds of Formula IIE are provided with the proviso that when z is two, R^{3b} is other than

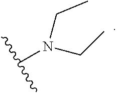 [SMILES: *N(CC)CC]

Compounds of Formula IIF are further provided as follows:

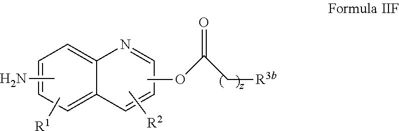

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

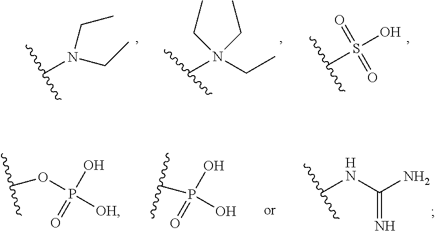

z=1−12;
and salts or solvates thereof.

Further provided herein are compounds of Formula IIG:

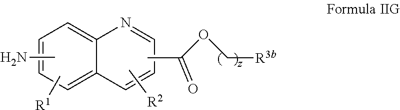

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

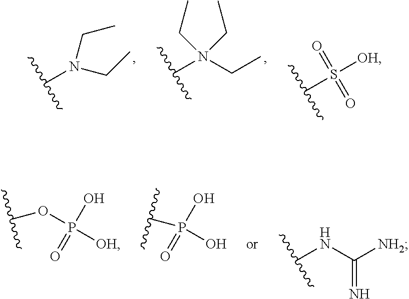

z=1−12;
and salts or solvates thereof.

In addition, provided below are exemplary compounds (Table A) of the structural Formulas II, IIA, IIB, IIC, IID, IIE, IIF or IIG which can be useful for fluorescent labeling of glycans and subsequent analysis by means of liquid chromatography and mass spectrometry. The compounds of structural Formulas II, IIA, IIB, IIC, IID, IIE, IIF or IIG can be optionally substituted with any group that does not substantially reduce the fluorescence of the compound.

TABLE A

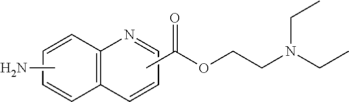

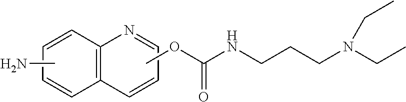

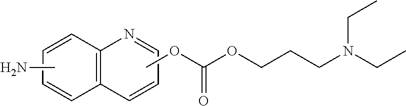

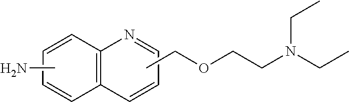

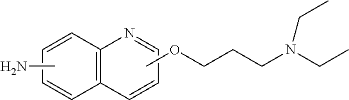

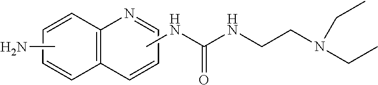

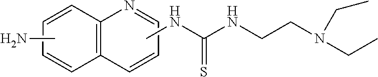

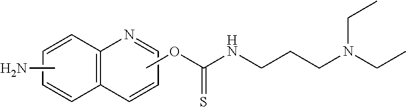

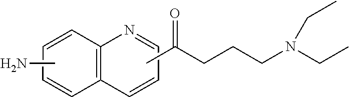

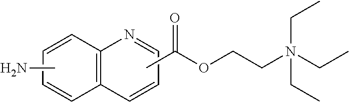

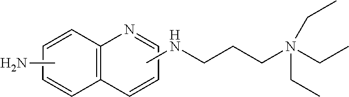

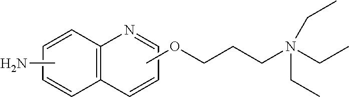

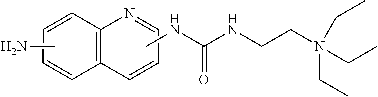

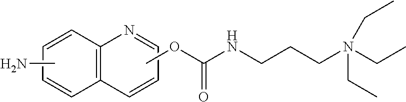

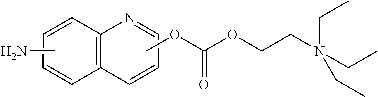

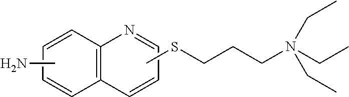

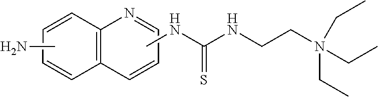

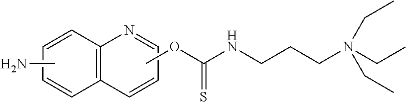

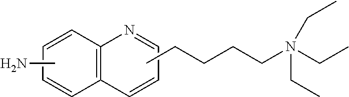

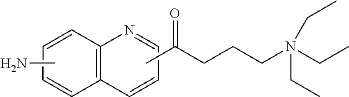

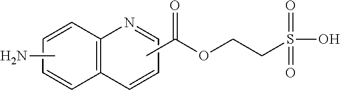

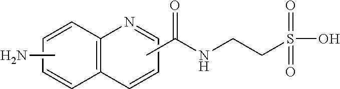

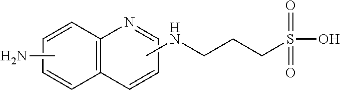

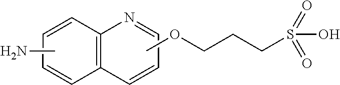

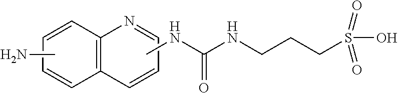

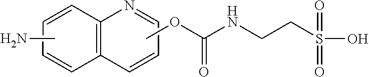

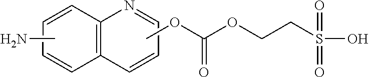

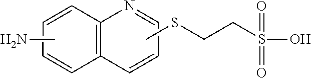

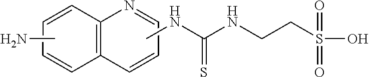

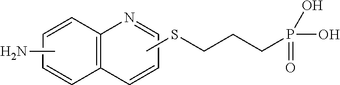

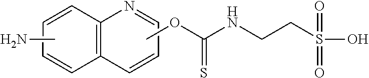

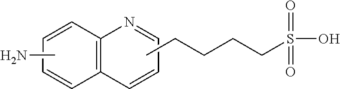

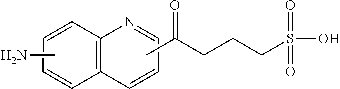

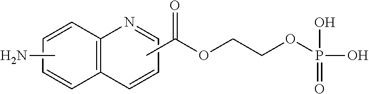

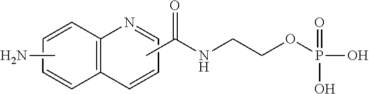

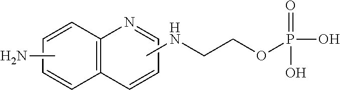

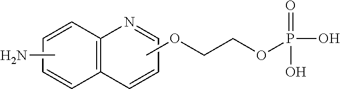

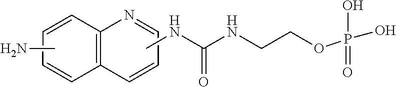

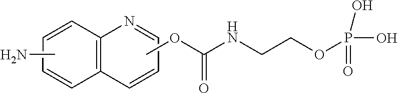

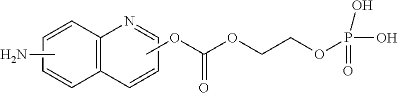

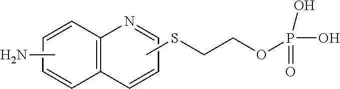

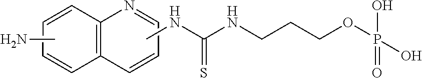

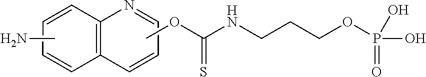

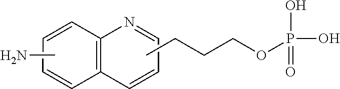

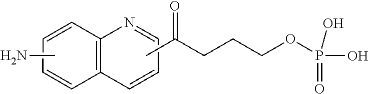

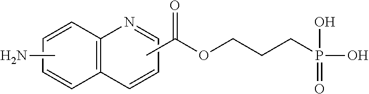

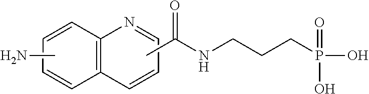

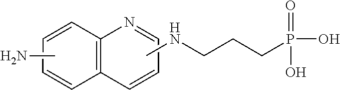

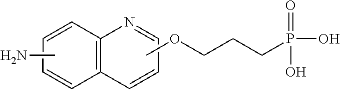

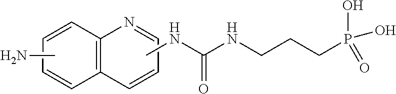

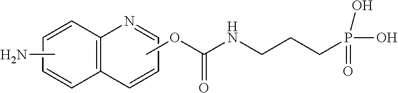

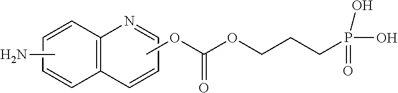

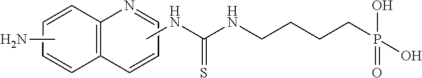

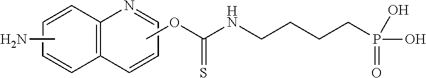

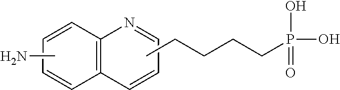

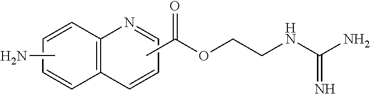

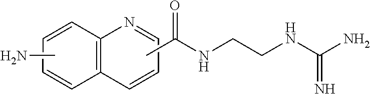

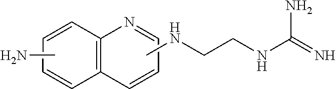

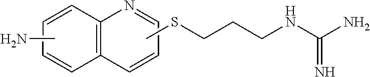

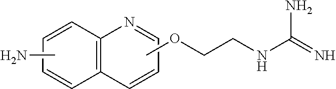

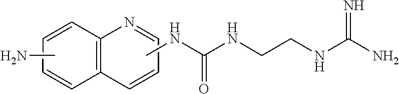

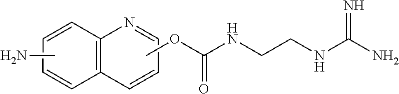

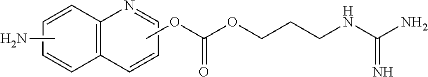

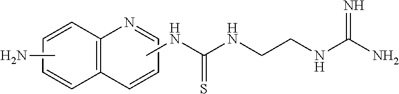

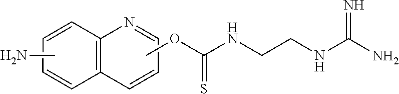

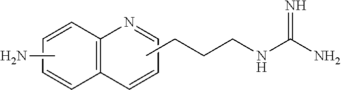

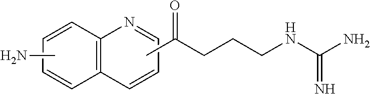

Provided herein are compounds of Formula II, IIA, IIB, IIC, IID, IIE, IIF or IIG wherein R¹ is hydrogen. In an embodiment, the compounds of Formula II, IIA, IIB, IIC, IID, IIE, IIF or IIG include compounds wherein R² is hydrogen. The compounds of Formula II, IIA, IIB, IIC, IID, IIE, IIF or IIG are also provided wherein R¹ and R² are hydrogen.

Methods for tagging, derivatizing or conjugating biomolecules containing at least one ketone group or aldehyde group with a compound of Formula II, IIA, IIB, IIC, IID, IIE, IIF or IIG, or a compound of Table A by reductive amination reaction are further provided. The reaction between a compound of Formula II and an aldehyde containing biomolecule, such as a glycan can be conducted under conditions that facilitate reductive amination in the presence of a reducing agent, such as from sodium cyanoborohydride or sodium triacetoxyborohydride. The reaction can be carried out in a solution or suspension of a compound of Formula II, IIA, IIB, IIC, IID, HIE, IIF or IIG, or a compound of Table A in an acidic media, for example in citric acid or acetic acid, and mixing with a solution or suspension of a reducing agent such as cyanoborohydride or sodium triacetoxyborohydride in an organic solvent, for example, tetrohydrofuran or dimethylsulfoxide.

Methods for analyzing a biomolecule containing an aldehyde group, such as a glycan, in a sample containing at least one biomolecule by means of liquid chromatography and mass spectrometry are provided. These methods comprise the step of labeling the biomolecule in the sample by reacting with a compound of Formula II, IIA, IIB, IIC, IID, IIE, IIF or IIG, or a compound of Table A for a time and under conditions suitable to facilitate the labeling through reductive amination; and subjecting the labeled conjugate to liquid chromatography and mass spectrometry.

Analytical kits for assaying glycans and other biomolecules comprise (i) a labeling module comprising a compound of Formula II and salts and solvate thereof; and optionally one or more of the following:
(i) a deglycosylation module comprising one or more endoglycosidases, a buffer, and one or more surfactants, or one or more compounds that can perform a chemical release of glycoprotein; and
(ii) a separation device for clean-up such as a solid phase extraction device, or a centrifugal filtration device or the like.

Solid phase extraction (“SPE”) is a sample preparation technology that utilizes solid particle, chromatographic packing material, usually contained in a cartridge type device, to chemically separate the different components of a sample. The SPE device having a chromatographic bed can perform four critical functions to make the analysis of the sample more successful including: (1) simplification of complex sample matrix along with compound purification; (2) reduction in ion suppression or enhancement in MS applications; (3) capability to fractionate sample matrix to analyze compounds by class; and (4) trace concentration enrichment of very low level compounds. In SPE, samples are typically in the liquid state although specialty applications may be run with some samples in the gas phase.

The separation device of the kit described herein, however, can include, but is not limited to, devices using reversed phase chromatography, ion exchange chromatography and hydrophilic interaction chromatography (“HILIC”) and include devices which utilize graphitic stationary phases such as porous graphitized carbon and mobile phases acidified by formic acid or are separated by capillary electrophoresis. In addition, desalting, buffer exchanges or diafiltration are methodologies associated with removing salts or solvents in solutions containing biomolecules. The removal of salts or the exchange of buffers can be accomplished in a centrifugal device such as the Amicon Ultra-0.5 device by concentrating the sample, then reconstituting the concentrate to the original sample volume with any desired solvent. The process of “washing out” can be repeated until the concentration of the contaminating microsolute has been sufficiently reduced. Noteworthy, as part of the kit, glycoproteins can be chemically deglycosylated through alkaline beta-elimination or hydrazinolysis as well as by endoglycosidases.

Glycans and other biomolecules can be conjugated to MS active fluorescent compounds of Formula II and salts or solvates thereof. The following schematic shows the tagging of a glycan using a compound of Formula II through reductive amination:

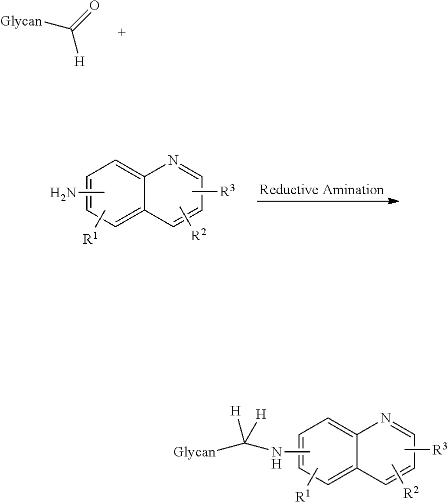

where FL R¹, R² and R³ are as described herein.

Therefore, methods of tagging biomolecules (also referred to herein sometimes as biomolecules), such as glycans, with the MS active fluorescent compounds of Formula II, as well as conjugates resulting therefrom are provided.

### Coumarin Based MS Active Fluorescence Tagging Compounds

The MS active, fluorescence tagging compounds can be a coumarin derivative of Formula III:

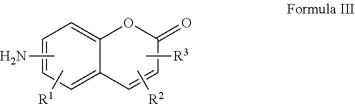

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R³ is

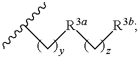

R^{3a} is selected from ester, amide, amine, oxygen, urea, carbamate, carbonate, sulfur, thiourea, thiocarbamate, alkyl or carbonyl;
R^{3b} is

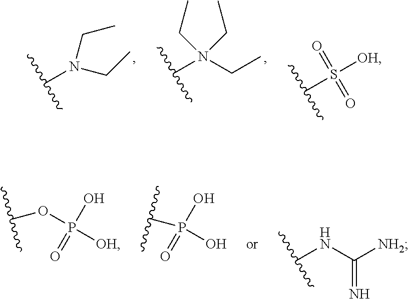

y=0−12;
z=1−12;
and salts or solvates thereof.

Provided herein are compounds of Formula III, with the proviso that when y is zero, R^{3a} is ester and z is two, R^{3b} is other than

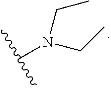 [SMILES: *N(CC)CC]

In an embodiment, the compounds of the structural Formula IIIA are:

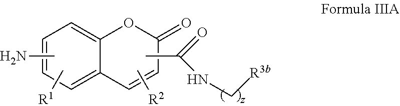

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

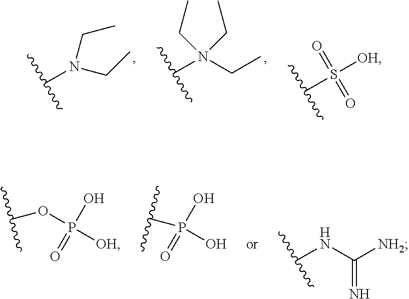

z=1−12;
and salts or solvates thereof.

In an embodiment, provided herein are compounds of Formula IIIB:

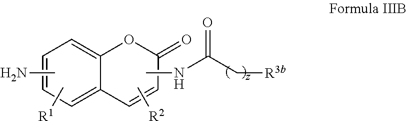

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

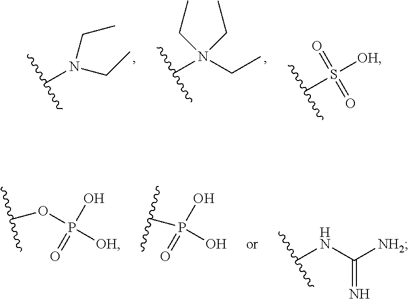

z=1−12;
and salts or solvates thereof.

In an embodiment, provided herein are compounds of Formula IIIC:

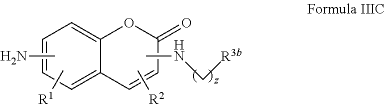

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

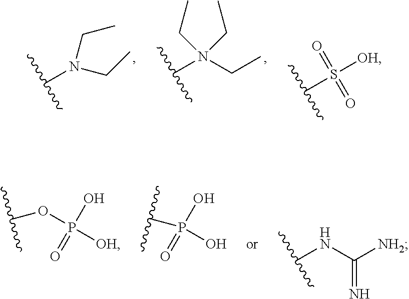

z=1−12;
and salts or solvates thereof.

In yet another embodiment, provided herein are compounds of the structural Formula IIID:

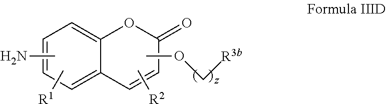

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

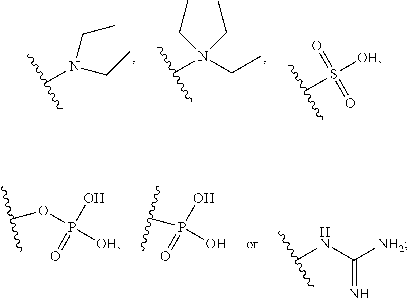

z=1−12;
and salts or solvates thereof.

In an embodiment, provided herein are compounds of the structural Formula IIIE:

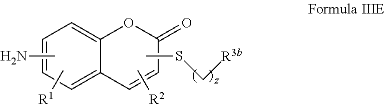

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

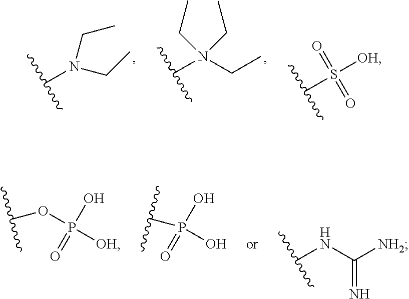

z=1−12;
and salts or solvates thereof.

In an embodiment, provided herein are compounds of structural Formula IIIF:

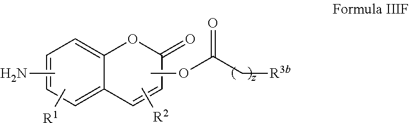

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

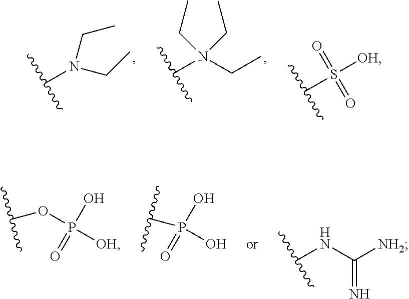

z=1−12;
and salts or solvates thereof.

In an embodiment, provided are compounds of Formula IIIG:

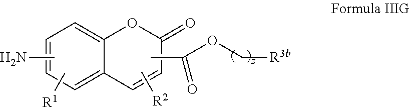

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

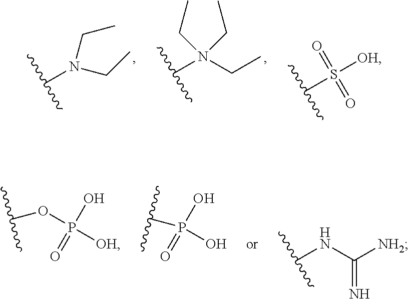

z=1−12;
and salts or solvates thereof.

In an embodiment, compounds are provided of the structural Formula IIIG, with the proviso that when y is zero, and z is two, R^{3b} is other than

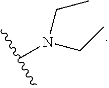 [SMILES: *N(CC)CC]

In addition, provided below are exemplary compounds (Table B) of the structural Formulas III, IIIA, IIIB, IIIC, IIID, IIIE, IIIF or IIIG which can be useful for fluorescent labeling of glycans and subsequent analysis by means of liquid chromatography and mass spectrometry. The compounds of structural Formulas III, IIIA, IIIB, IIIC, IIID, IIIE, IIIF or IIIG could be optionally substituted with any group that does not substantially reduce the fluorescence of the compound.

TABLE B

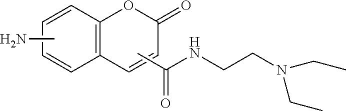

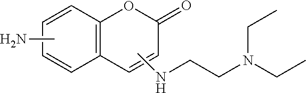

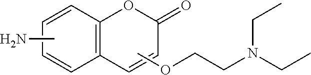

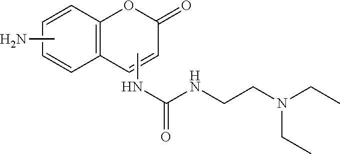

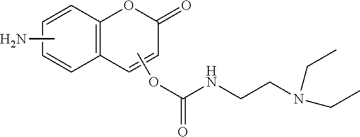

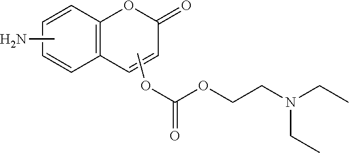

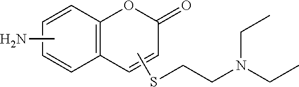

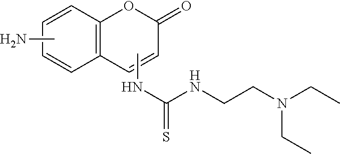

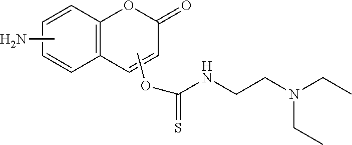

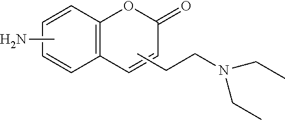

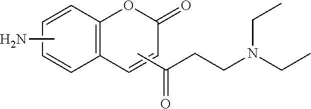

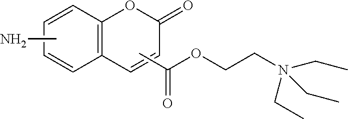

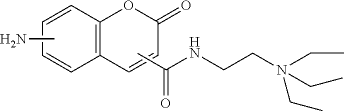

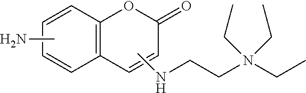

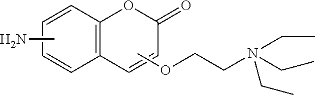

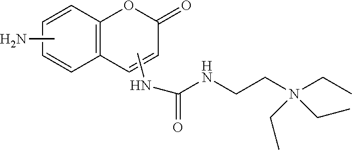

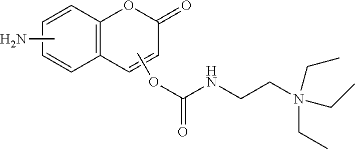

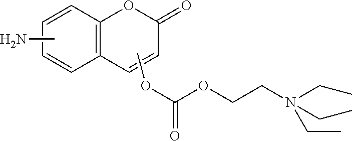

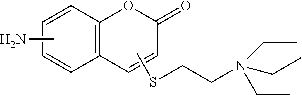

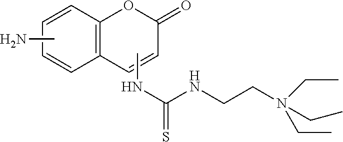

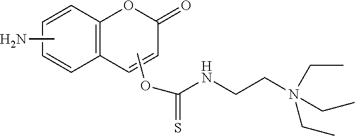

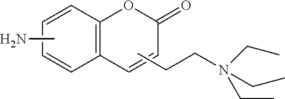

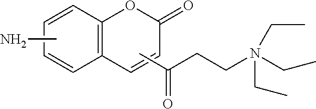

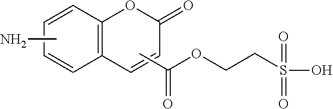

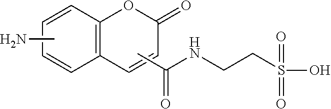

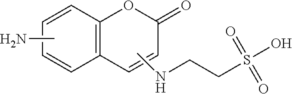

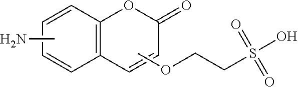

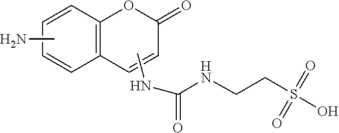

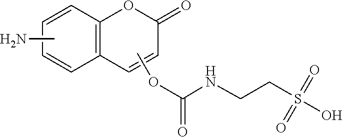

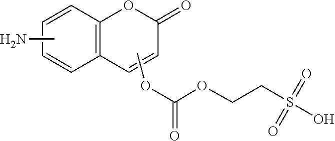

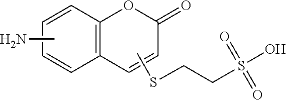

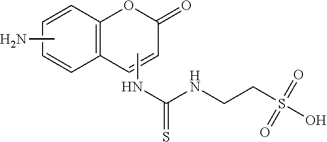

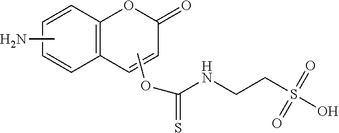

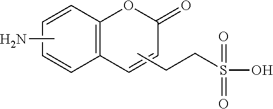

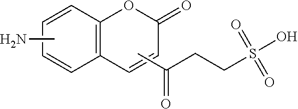

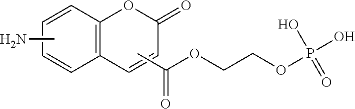

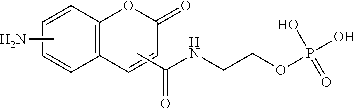

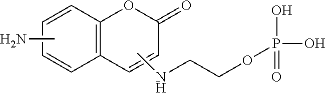

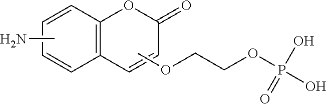

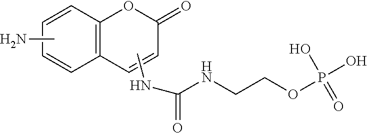

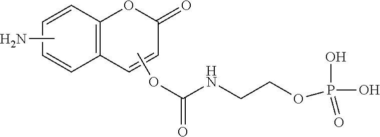

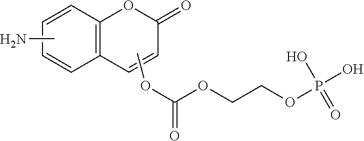

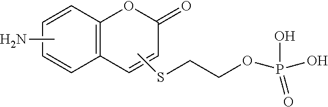

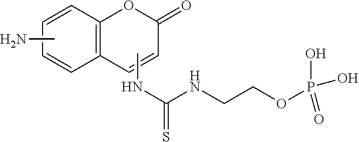

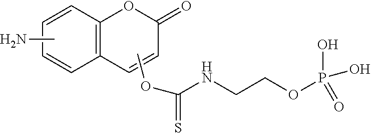

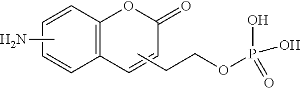

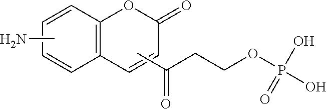

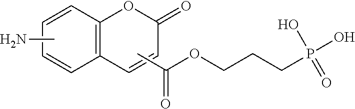

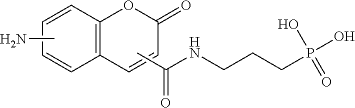

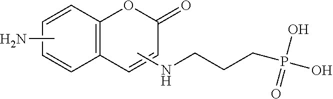

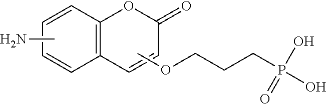

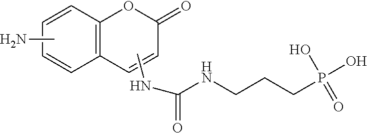

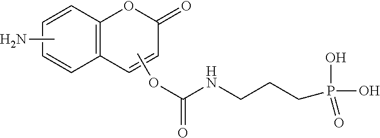

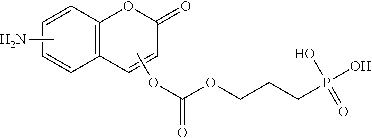

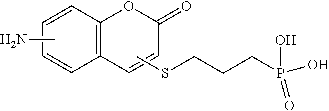

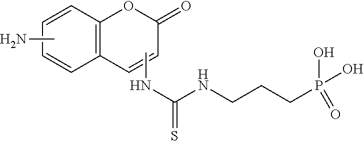

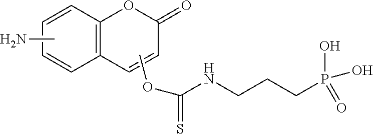

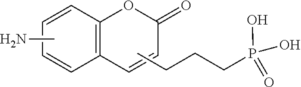

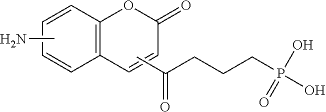

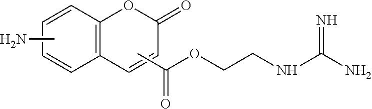

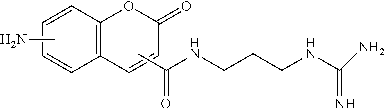

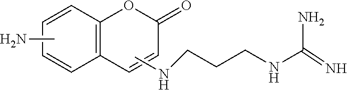

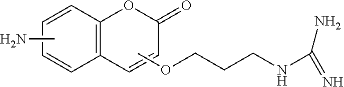

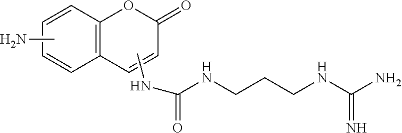

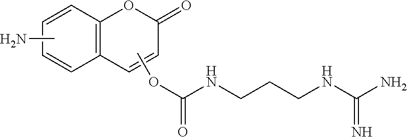

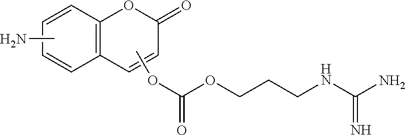

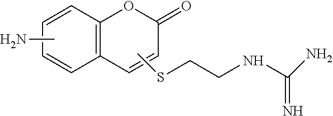

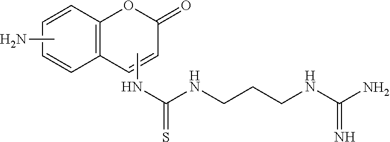

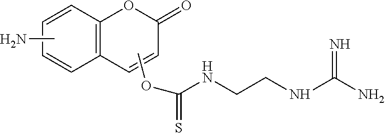

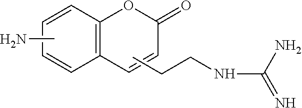

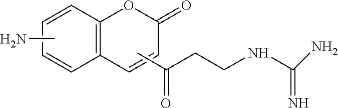

In an embodiment, provided herein are compounds of Formula III, IIIA, IIIB, IIIC, IIID, IIIE, IIIF or IIIG wherein R¹ is hydrogen. In an embodiment, provided herein are compounds of Formula III, IIIA, IIIB, IIIC, IIID, IIIE, IIIF or IIIG wherein R² is hydrogen.

In an embodiment, provided herein are compounds of Formula III, IIIA, IIIB, IIIC, IIID, IIIE, IIIF or IIIG wherein R¹ and R² are hydrogen.

Methods for tagging, derivatizing or conjugating glycans and other biomolecules containing at least one ketone group or an aldehyde group with a compound of Formula III, IIIA, IIIB, IIIC, IIID, IIIE, IIIF or IIIG or a compound of Table B by reductive amination reaction are also provided. The reaction between a compound of Formula III and an aldehyde containing biomolecule, such as a glycan can be conducted under conditions that facilitate reductive amination in the presence of a reducing agent selected from sodium cyanoborohydride or sodium triacetoxyborohydride. The reaction can be carried out in a solution or suspension of a compound of Formula III, IIIA, IIIB, IIIC, IIID, IIIE, IIIF or IIIG or a compound of Table B in an acidic media, for example in citric acid or acetic acid, and mixing with a solution or suspension of a reducing agent such as cyanoborohydride or sodium triacetoxyborohydride in an organic solvent, for example, tetrohydrofuran or dimethylsulfoxide.

Further provided are methods for analyzing a biomolecule containing an aldehyde group, such as a glycan, in a sample containing at least one biomolecule, such as a glycan, by means of liquid chromatography and mass spectrometry. The analytical method comprises the steps of labeling the biomolecule, such as a glycan, in the sample by reacting with a compound of Formula III, IIIA, IIIB, IIIC, IIID, IIIE, IIIF or IIIG or a compound of Table B for a time and under conditions suitable to facilitate the labeling through reductive amination; and subjecting the labeled conjugate to liquid chromatography and mass spectrometry.

Analytical kits for assaying biomolecules, such as glycans, are provided where the kits comprise (i) a labeling module comprising a compound of Formula III and salts and solvate thereof; and optionally one or more of the following:
(i) a deglycosylation module comprising one or more endoglycosidases, a buffer, and one or more surfactants, or one or more compounds that can perform a chemical release of glycoprotein; and
(ii) a separation device for clean-up such as a solid phase extraction device or a centrifugal filtration device or the like.

Biomolecules, such as glycans, can be conjugated to MS active fluorescent compounds of Formula III and salts or solvates thereof. The following schematic shows the tagging of a glycan using a compound of Formula III through reductive amination:

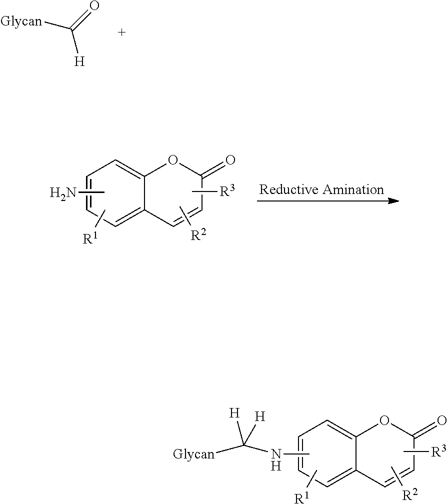

wherein FL R¹, R² and R³ are as described above

Hence, the methods of tagging glycans, with MS active fluorescent compounds of Formula III, as well as conjugates resulting therefrom are provided.

### Naphthalene Based MS Active Fluorescence Tagging Compounds

The MS active, fluorescence tagging compounds can be a naphthalene derivative of Formula IV:

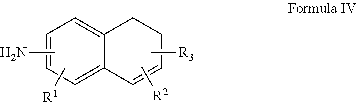

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R³ is

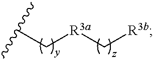

R^{3a} is selected from ester, amide, amine, oxygen, urea, carbamate, carbonate, sulfur, thiourea, thiocarbamate, alkyl or carbonyl;
R^{3b} is

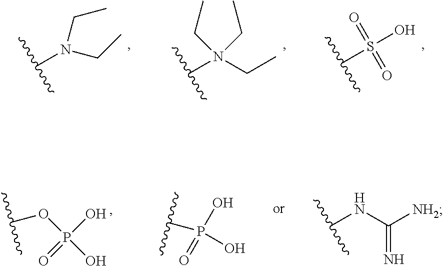

y=0−12;
z=1−12;
and salts or solvates thereof.

In an embodiment, provided herein are compounds of Formula IV, with the proviso that when y is two, R^{3a} is other than ester.

In an embodiment, provided herein are compounds of Formula IV, with the proviso that when y is zero, R^{3a} is oxygen, amide or ester, and z is two or three, R^{3b} is other than

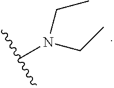 [SMILES: *N(CC)CC]

In an embodiment, provided herein are compounds of Formula IV, with the proviso that when y is zero and R^{3a} is oxygen or amine, and z is three, R^{3b} is other than —S(O)₃H.

In an embodiment, provided herein are compounds of Formula IVA:

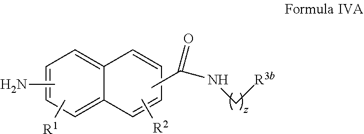

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

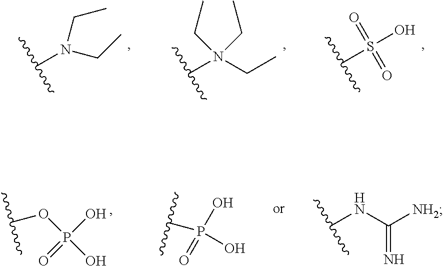

z=1−12;
and salts or solvates thereof.

In an embodiment, provided herein are compounds of Formula IVB:

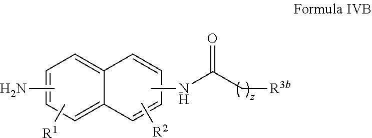

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

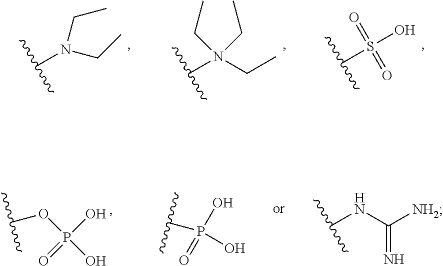

z=1−12;
and salts or solvates thereof.

In an embodiment, provided are compounds of Formula IVC:

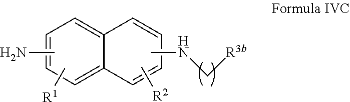

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

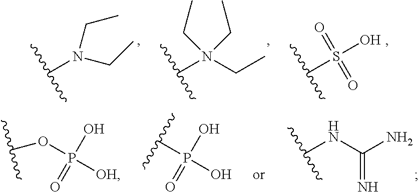

z=1−12;
and salts or solvates thereof.

In an embodiment, provided are compounds of Formula IVC with the proviso that when z is two, R^{3b} is other than

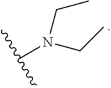 [SMILES: *N(CC)CC]

In an embodiment, provided are compounds of Formula IVD:

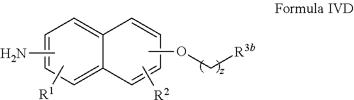

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

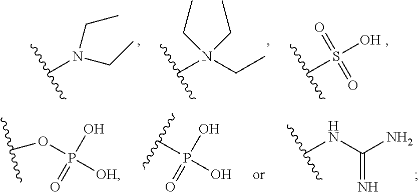

z=1−12;
and salts or solvates thereof.

In an embodiment, provided are compounds of Formula IVD with the proviso that when z is two, R^{3b} is other than

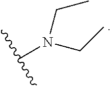 [SMILES: *N(CC)CC]

In an embodiment, provided are compounds of Formula IVE:

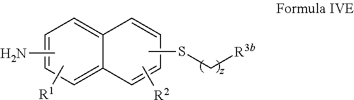

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

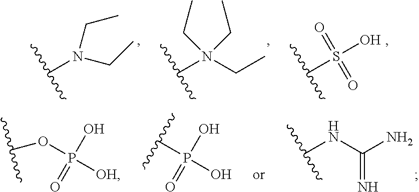

z=1−12;
and salts or solvates thereof.

In an embodiment, provided are compounds of structural Formula IVE with the proviso that when z is two, R^{3b} is other than

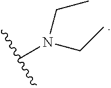 [SMILES: *N(CC)CC]

In an embodiment, provided are compounds of Formula IVF:

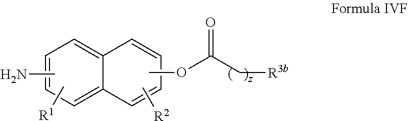

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

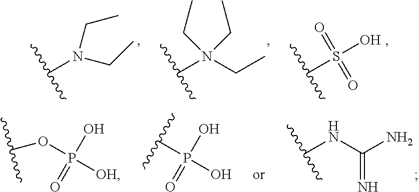

z=1−12;
and salts or solvates thereof.

In an embodiment, provided are compounds of Formula IVG:

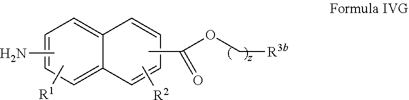

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

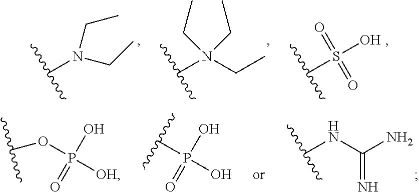

z=1−12;
and salts or solvates thereof.

In addition, provided below are exemplary compounds (Table C) of the structural Formulas IV, IVA, IVB, IVC, IVD, IVE, IVF or IVG which can be useful for fluorescent labeling of glycans and subsequent analysis by means of liquid chromatography and mass spectrometry. The compounds of structural formulas IV, IVA, IVB, IVC, IVD, IVE, IVF or IVG can be optionally substituted with any group that does not substantially reduce the fluorescence of the compound.

TABLE C

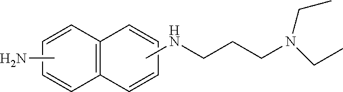

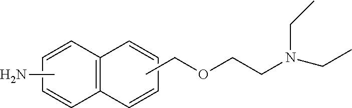

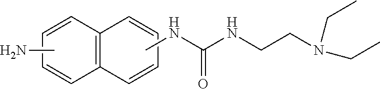

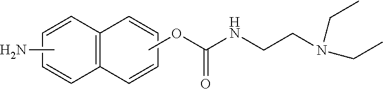

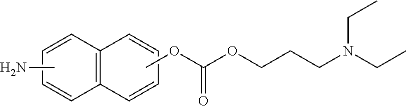

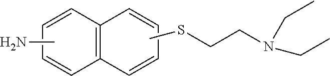

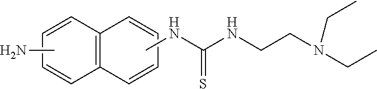

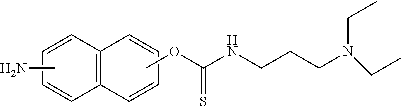

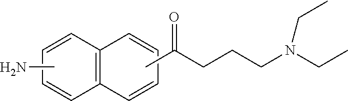

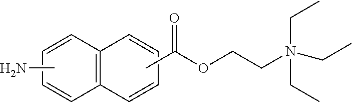

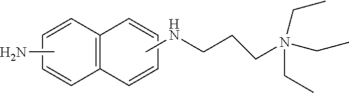

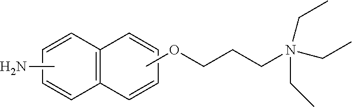

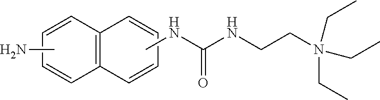

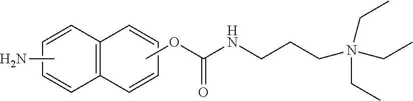

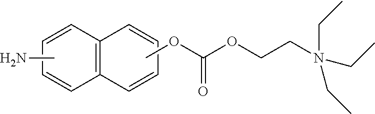

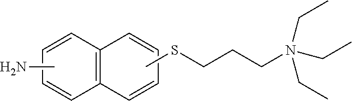

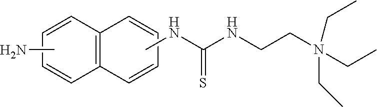

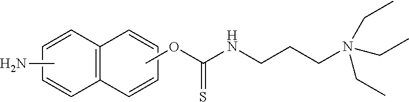

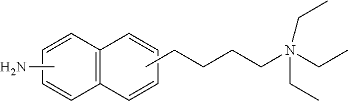

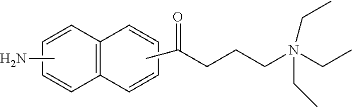

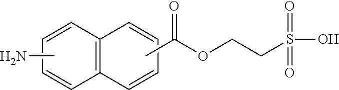

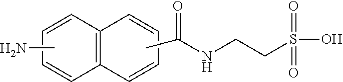

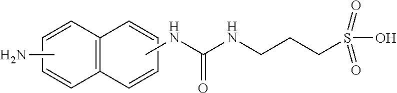

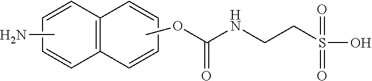

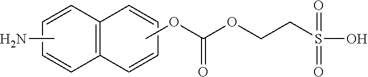

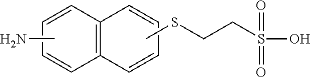

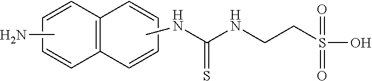

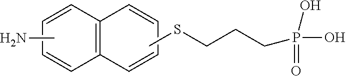

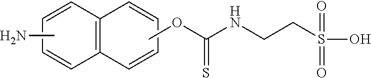

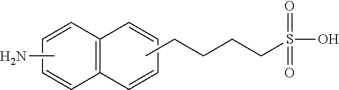

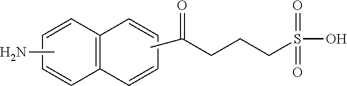

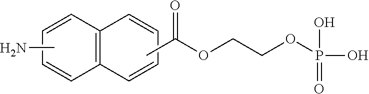

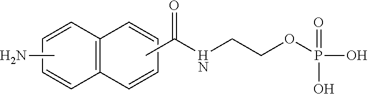

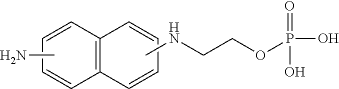

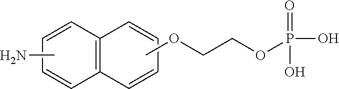

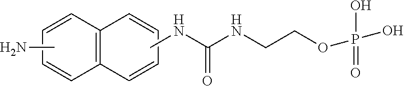

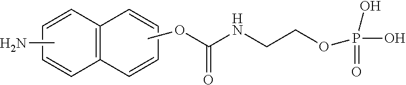

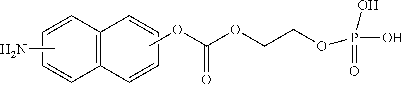

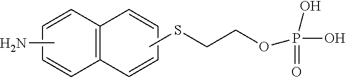

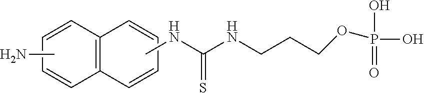

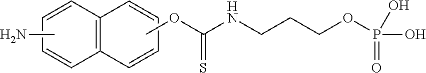

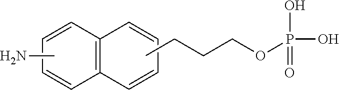

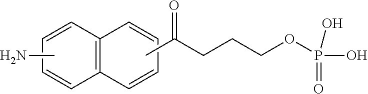

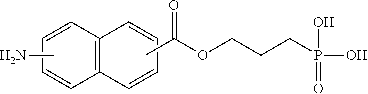

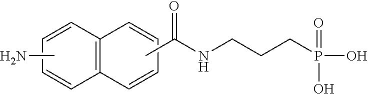

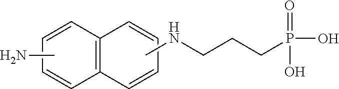

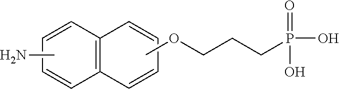

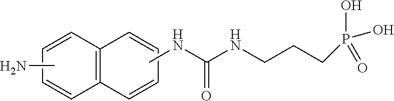

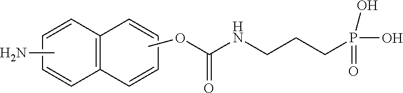

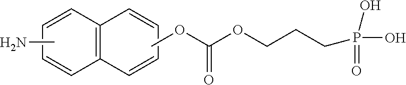

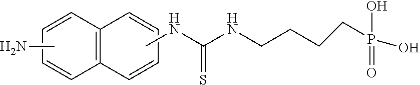

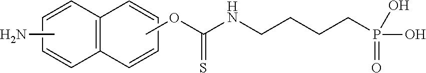

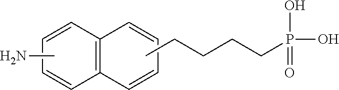

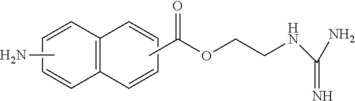

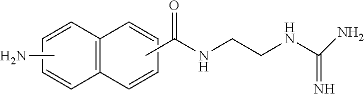

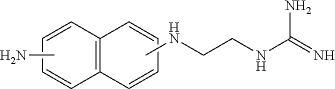

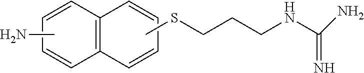

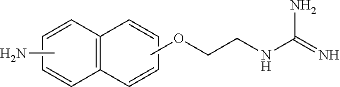

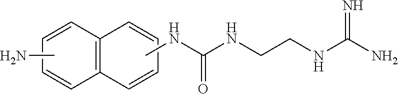

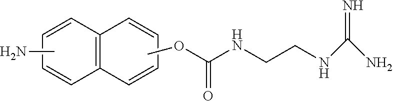

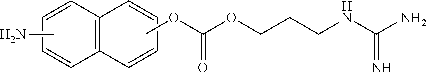

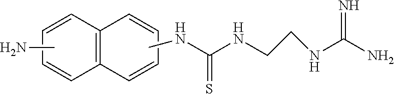

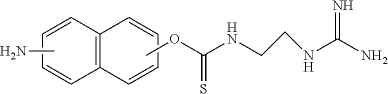

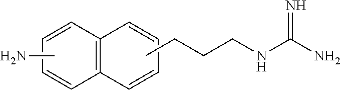

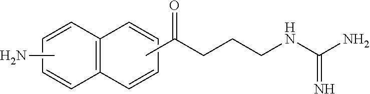

In an embodiment, provided herein are compounds of Formula IV, IVA, IVB, IVC, IVD, IVE, IVF or IVG wherein R¹ is hydrogen. In an embodiment, provided herein are compounds of Formula IV, IVA, IVB, IVC, IVD, IVE, IVF or IVG wherein R² is hydrogen. In an embodiment, provided herein are compounds of Formula IV, IVA, IVB, IVC, IVD, IVE, IVF or IVG wherein R¹ and R² are hydrogen.

The methods for tagging, derivatizing or conjugating glycans and other biomolecules containing at least one ketone group or an aldehyde group are also provided with a compound of Formula IV, IVA, IVB, IVC, IVD, IVE, IVF or IVG by reductive amination reaction are also provided. The reaction between a compound of Formula IV and an aldehyde containing biomolecule, such as a glycan can be conducted under conditions that facilitate reductive amination in the presence of a reducing agent, such as sodium cyanoborohydride or sodium triacetoxyborohydride. The reaction can be carried out in a solution or suspension of a compound of Formula IV, IVA, IVB, IVC, IVD, IVE, IVF or IVG in an acidic media, for example in citric acid or acetic acid, and mixing with a solution or suspension of a reducing agent such as cyanoborohydride or sodium triacetoxyborohydride in an organic solvent, for example, tetrohydrofuran or dimethylsulfoxide.

Methods for analyzing a biomolecule containing an aldehyde group, such as a glycan, in a sample containing at least one biomolecule, such as a glycan, by means of liquid chromatography and mass spectrometry are also provided. The analytical methods comprise the steps of labeling the biomolecule, such as a glycan, in the sample by reacting with a compound of Formula IV, IVA, IVB, IVC, IVD, IVE, IVF or IVG for a time and under conditions suitable to facilitate the labeling through reductive amination; and subjecting the labeled conjugate to liquid chromatography and mass spectrometry.

Analytical kits for assaying biomolecules, such as glycans, can include (i) a labeling module comprising a compound of Formula IV and salts and solvate thereof, and optionally one or more of the following:
(i) a deglycosylation module comprising one or more endoglycosidases, a buffer, and one or more surfactants, or one or more compounds that can perform a chemical release of glycoprotein; and
(ii) a separation device for clean-up such as a solid phase extraction device or a centrifugal filtration device or the like.

Glycans can be conjugated to MS active fluorescent compounds of Formula IV and salts or solvates thereof. The following schematic shows the tagging of a glycan using a compound of Formula IV through reductive amination:

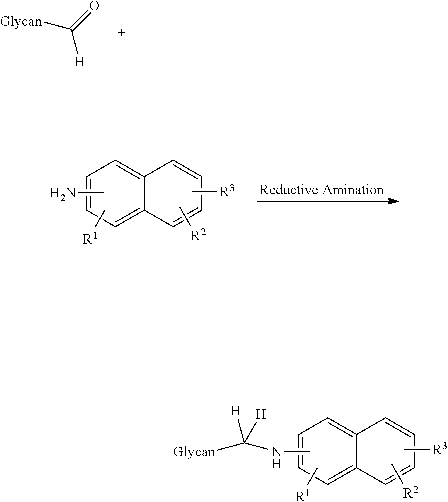

wherein FL R¹, R² and R³ are as described above.

Therefore, methods of tagging glycans with MS active fluorescent compounds of Formula IV, as well as conjugates resulting therefrom are provided herein.

### Rhodamine Based MS Active Fluorescence Tagging Compounds

The MS active, fluorescence tagging compounds can be a rhodamine derivative of Formula V, VI, VII, VIII or IX:

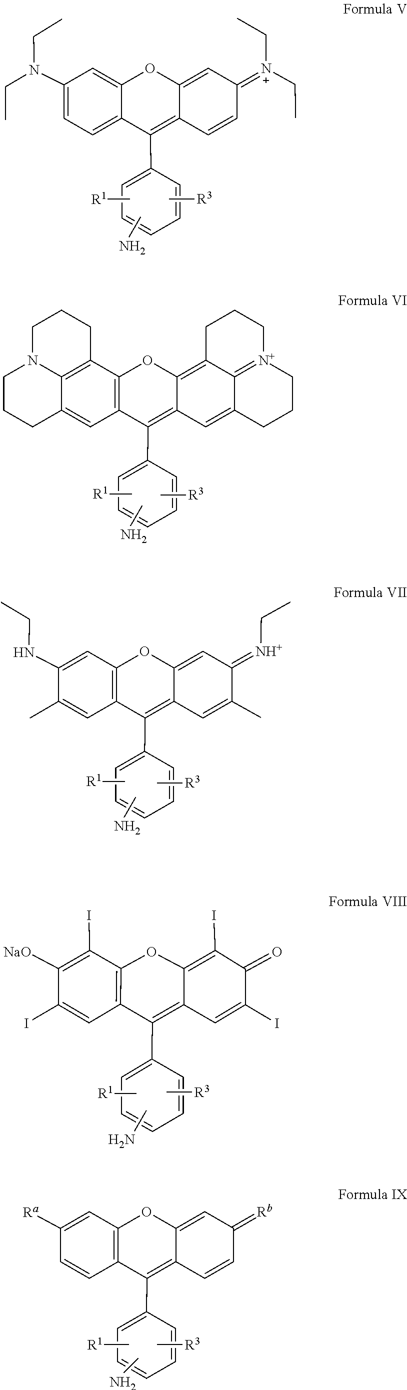

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R³ is

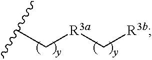

R^{3a} is selected from ester, amide, amine, oxygen, urea, carbamate, carbonate, sulfur, thiourea, thiocarbamate, alkyl or carbonyl;
R^{3b} is

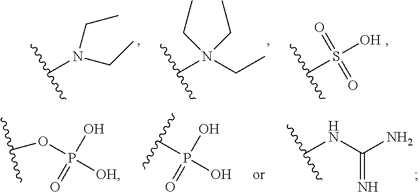

y=0−12;
z=1−12;
R^{a} is selected from

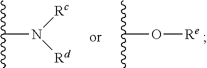

R^{b} is oxo or;

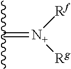

R^{c}, R^{d}, R^{e}, R^{f} and R^{g} are independently selected from hydrogen and optionally substituted alkyl; and salts or solvates thereof.

In an embodiment, provided herein are compounds of Formula VA, VIA, VIIA, VIIIA or IXA:

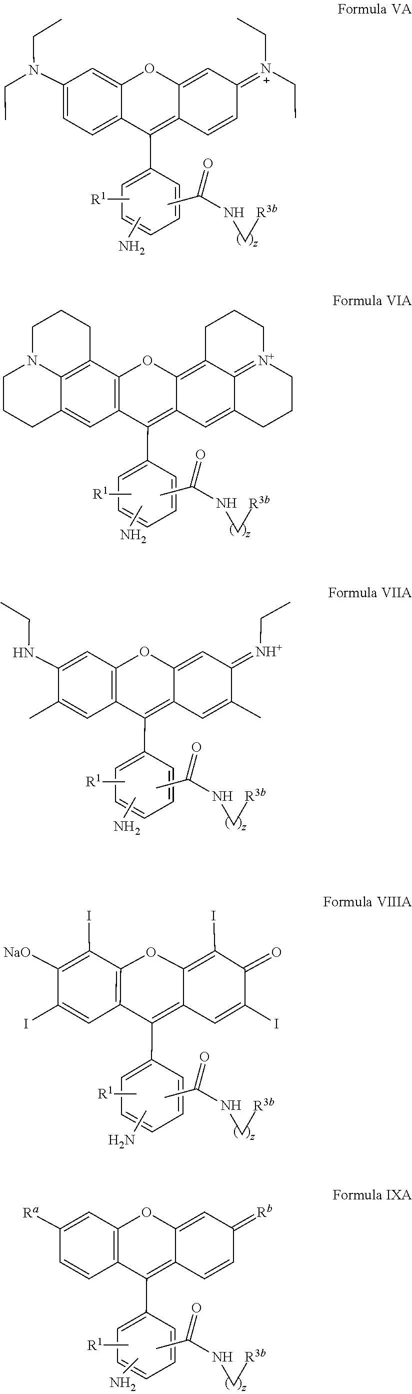

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

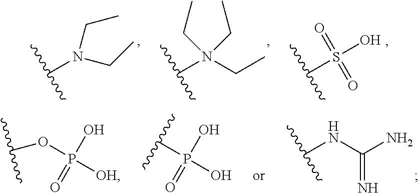

z=1−12;
R^{a} is selected from

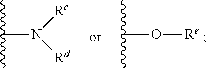

R^{b} is oxo or

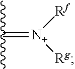

R^{c}, R^{d}, R^{e}, R^{f} and R^{g} are independently selected from hydrogen and optionally substituted alkyl; and salts or solvates thereof.

In an embodiment, provided herein are compounds of Formula VB, VIB, VIIB, VIIIB or IXB:

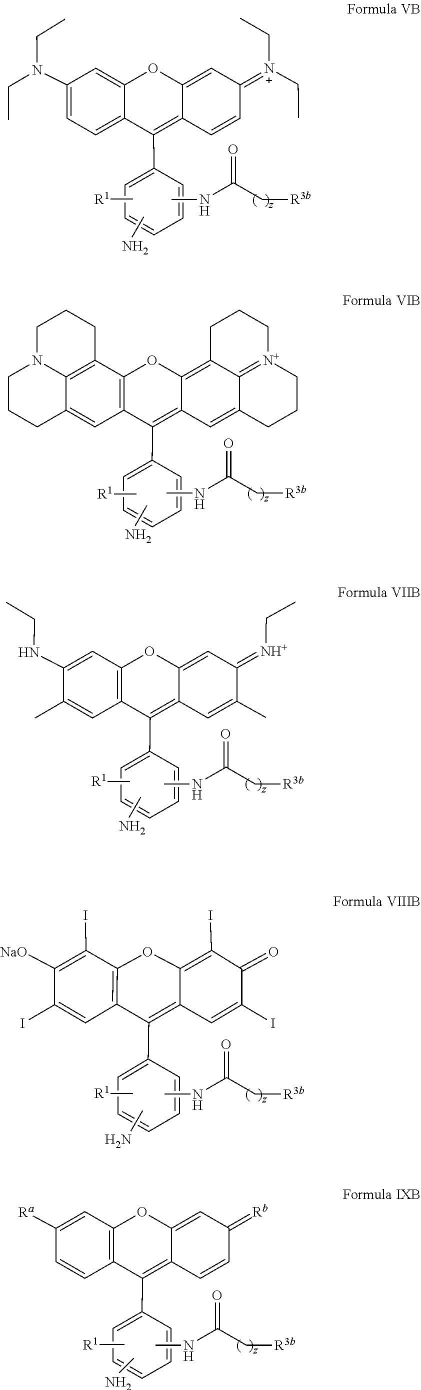

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

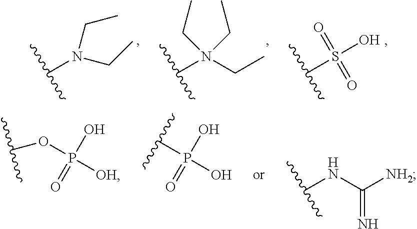

z=1−12;
R^{a} is selected from

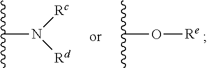

R^{b} is oxo or

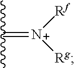

R^{c}, R^{d}, R^{e}, R^{f} and R^{g} are independently selected from hydrogen and optionally substituted alkyl; and salts or solvates thereof.

In yet another embodiment, provided are compounds of Formula VC, VIC, VIIC, VIIIC or IXC:

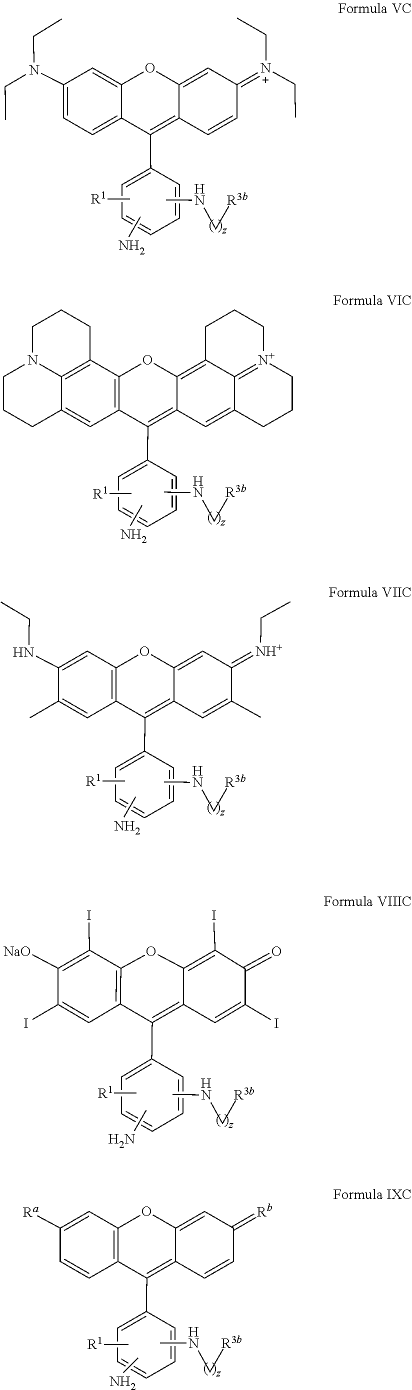

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

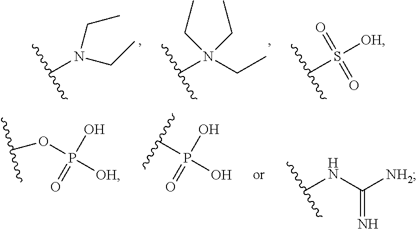

z=1−12;
and salts or solvates thereof.

In an embodiment, provided herein are compounds of Formula VD, VID, VIID, VIIID or IXD:

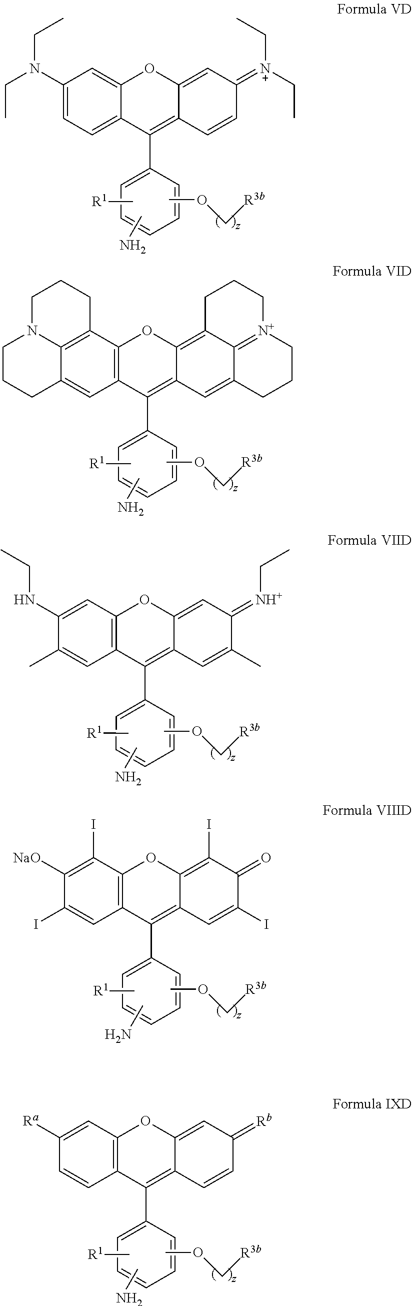

In an embodiment, provided herein are compounds of Formula VE, VIE, VIIE, VIIIE or IXE:

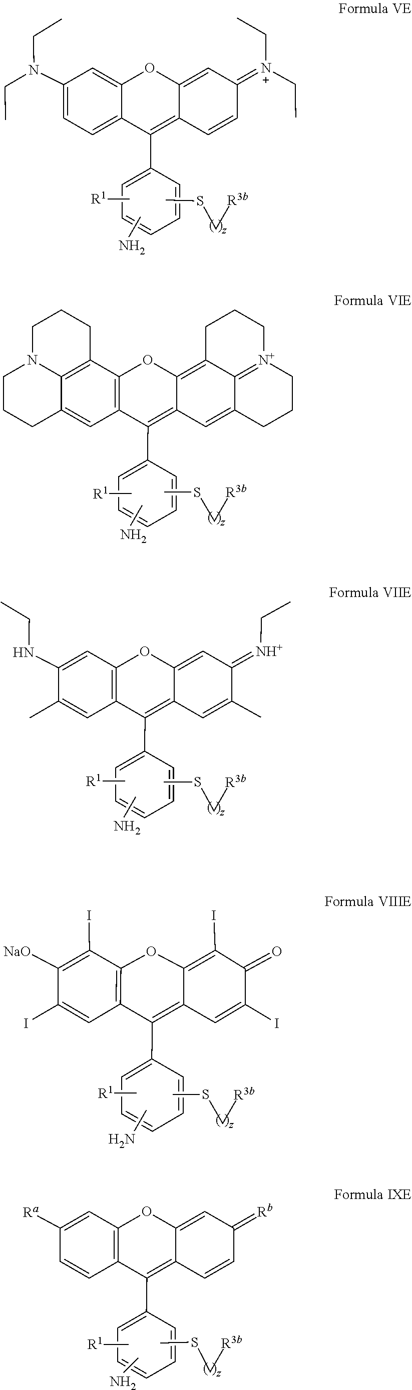

wherein each R₁ and R₂ is independently selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

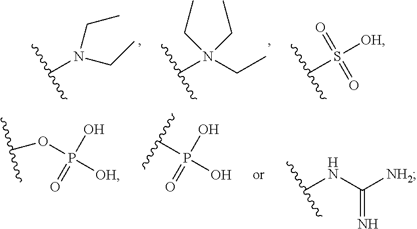

z=1−12;
and salts or solvates thereof.

In addition, provided below are exemplary compounds (Table D) of the structural Formulas V, VI, VII, VIII and IX which can be useful for fluorescent labeling of glycans and subsequent analysis by means of liquid chromatography and mass spectrometry. The compounds of structural Formulas V, VI, VII, VIII and IX can be optionally substituted with any group that does not substantially reduce the fluorescence of the compound.

TABLE D

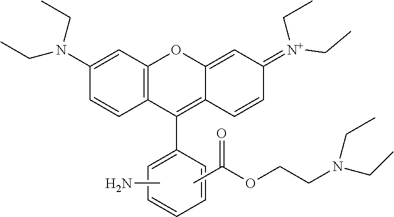

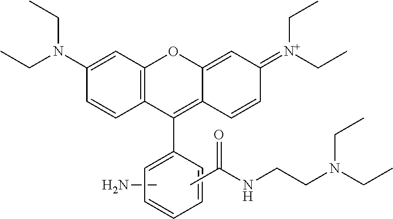

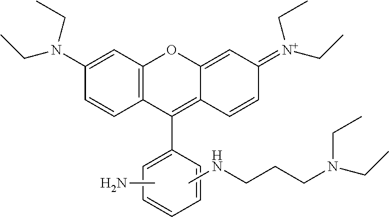

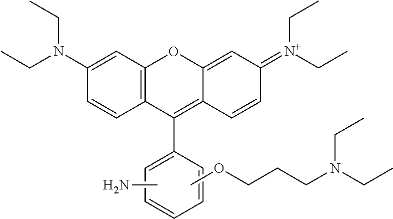

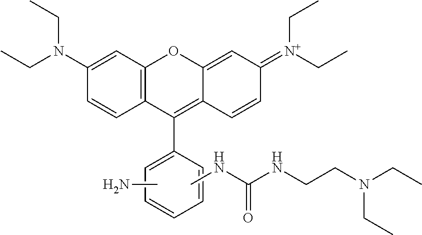

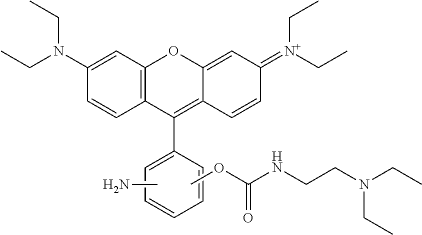

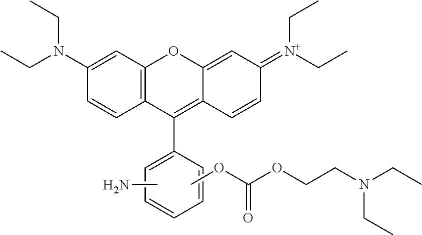

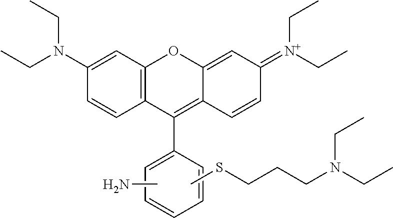

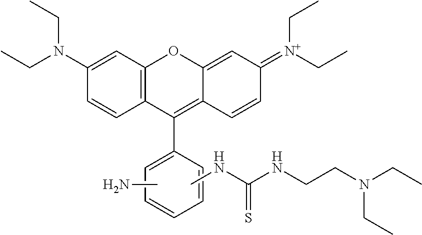

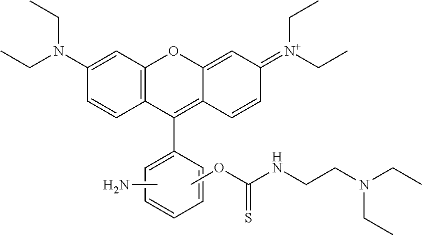

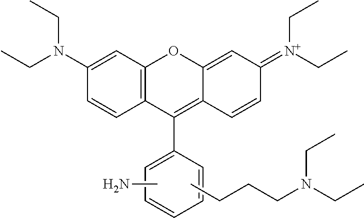

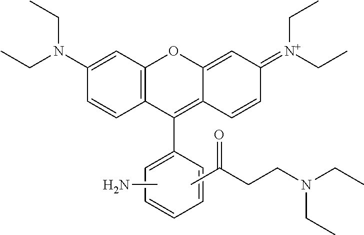

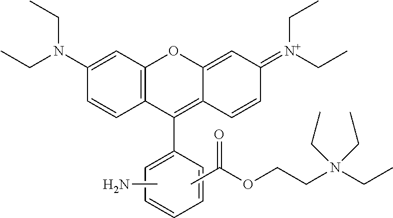

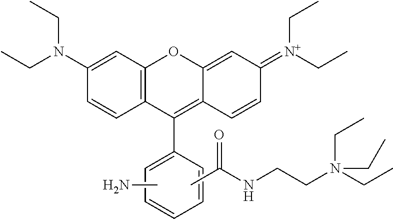

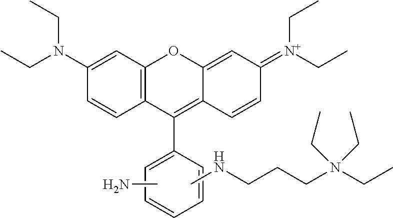

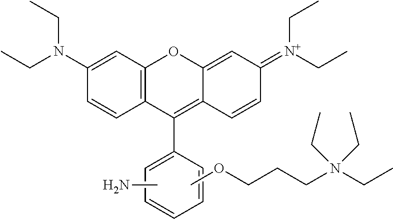

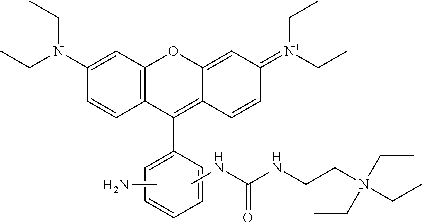

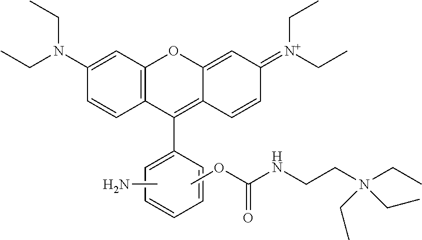

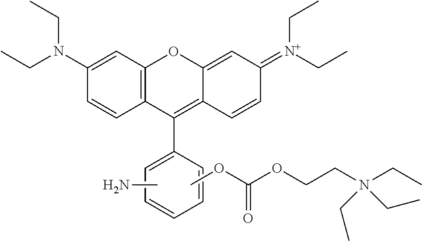

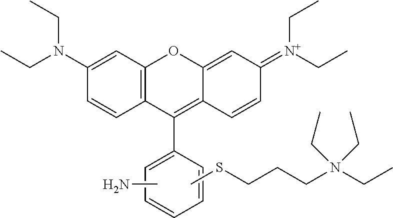

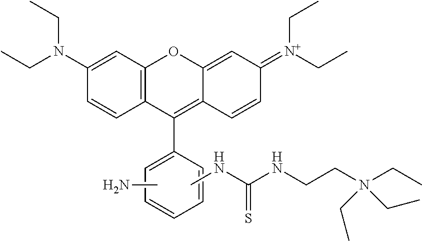

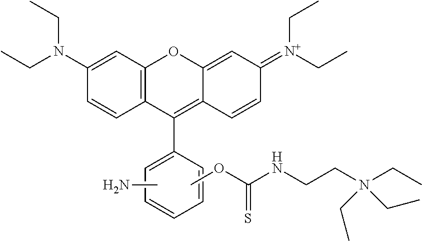

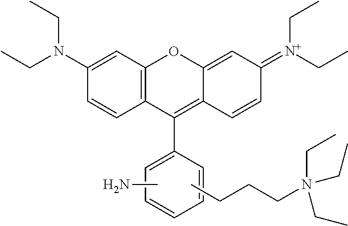

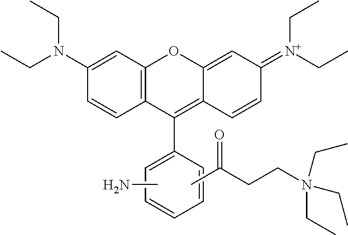

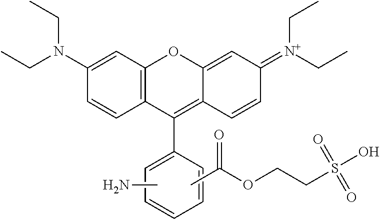

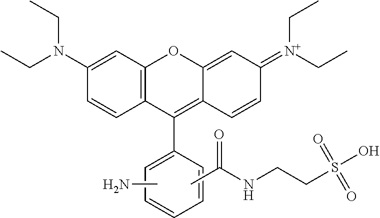

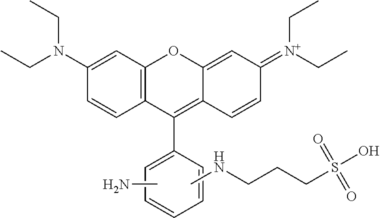

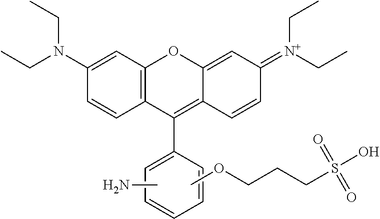

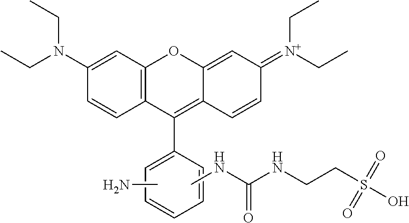

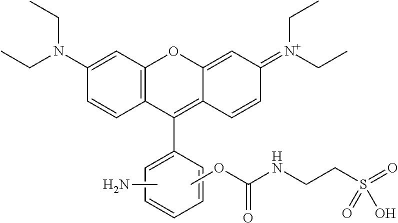

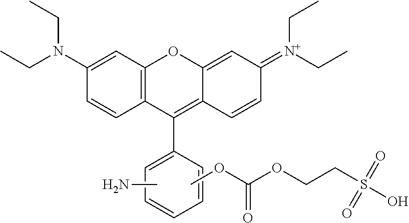

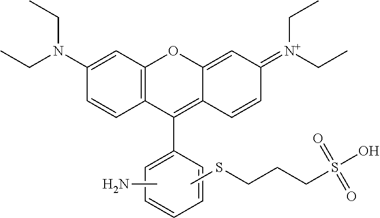

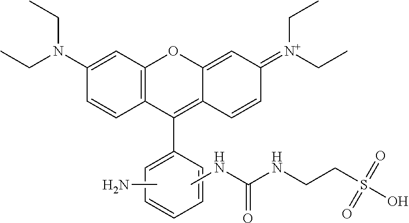

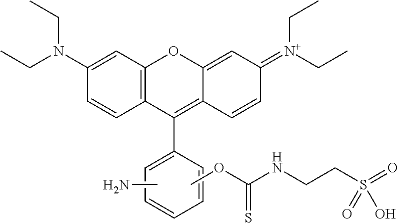

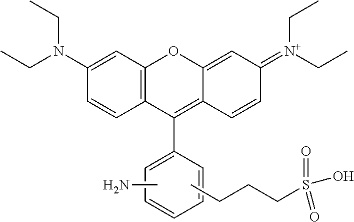

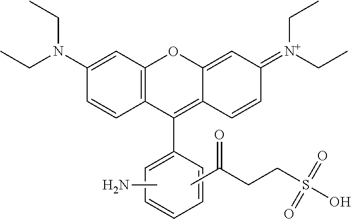

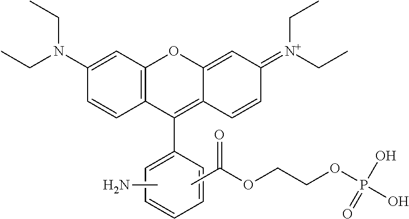

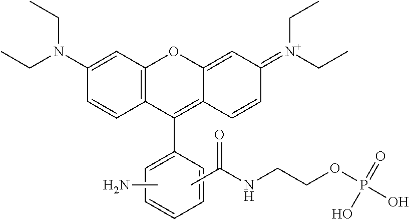

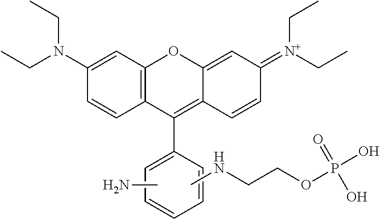

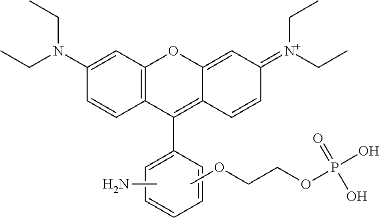

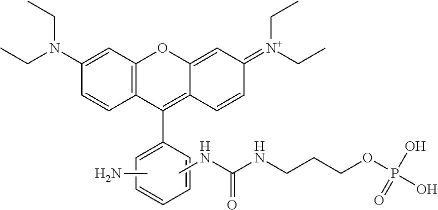

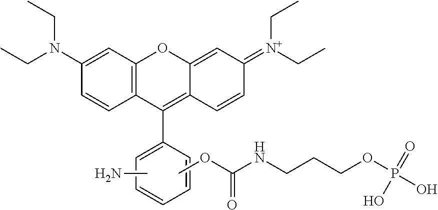

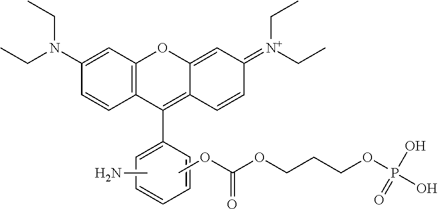

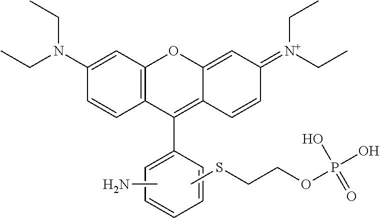

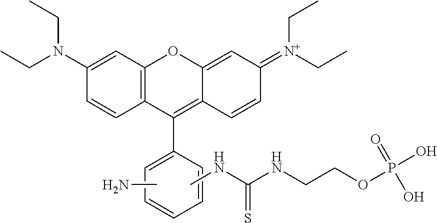

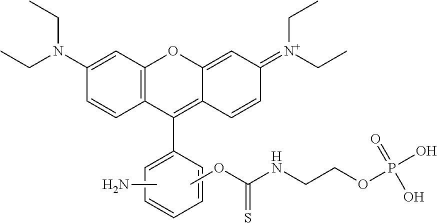

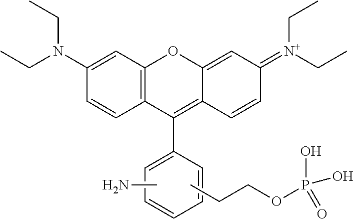

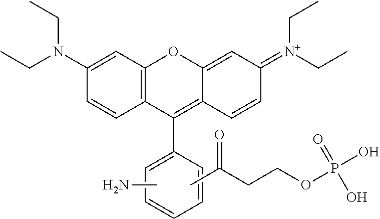

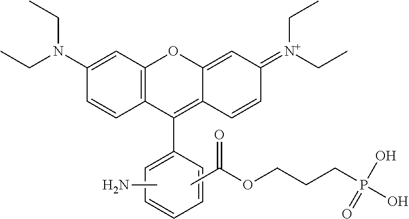

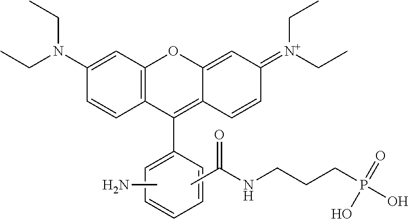

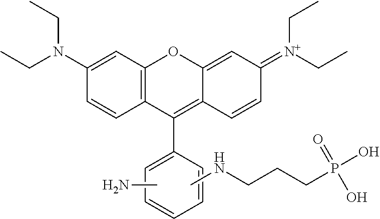

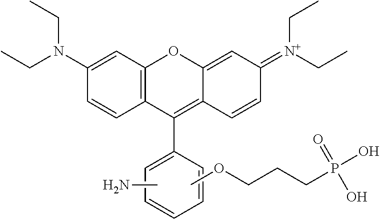

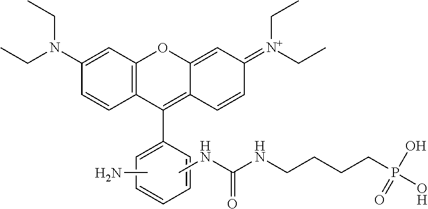

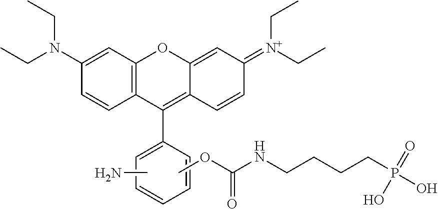

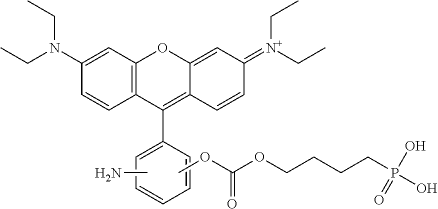

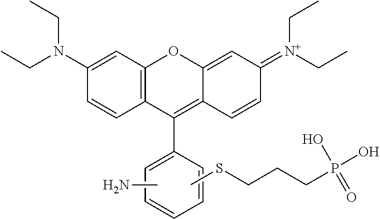

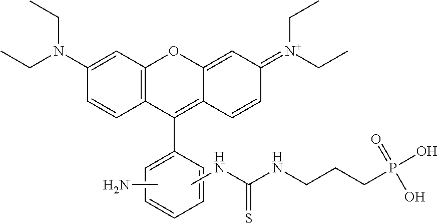

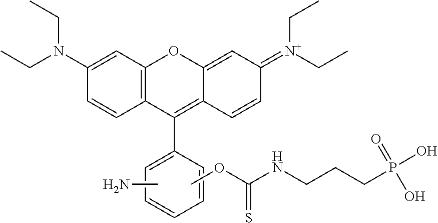

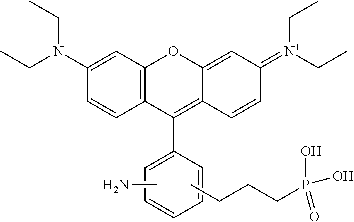

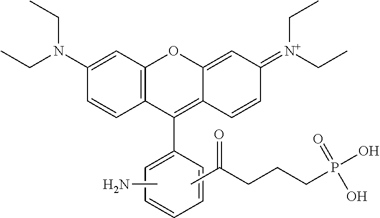

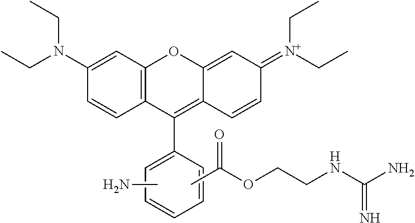

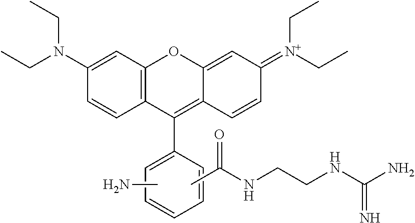

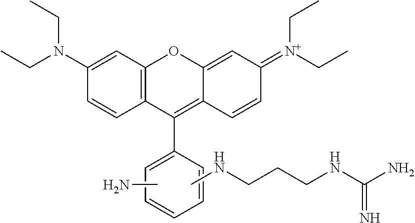

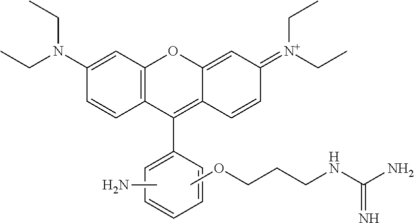

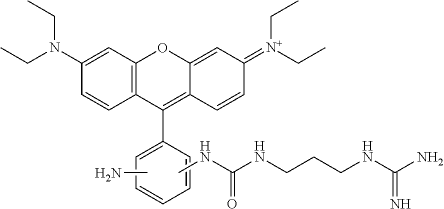

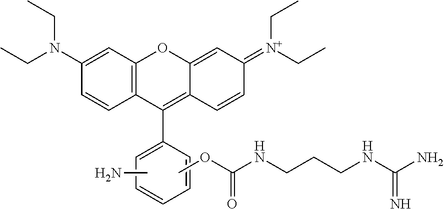

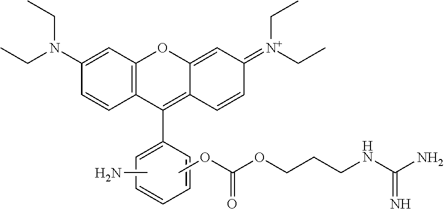

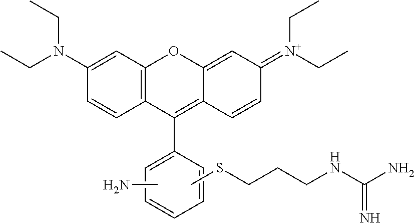

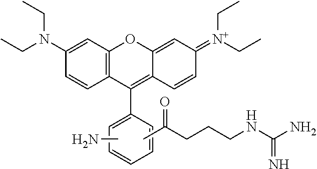

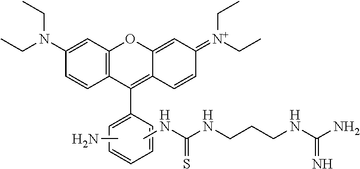

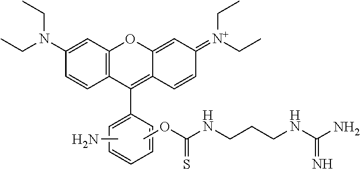

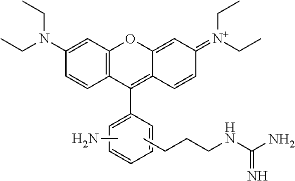

The compounds of Formula V, VI, VII, VIII or IX are provided wherein R¹ is hydrogen. In an embodiment, provided herein are compounds of Formula V, VI, VII, VIII or IX wherein R² is hydrogen. In an embodiment, provided herein are compounds of Formula V, VI, VII, VIII or IX wherein R¹ and R² are hydrogen.

Methods for tagging, derivatizing or conjugating glycans and other biomolecules containing at least one ketone group or an aldehyde group with a compound of Formula V, VI, VII, VIII or IX or a compound of Table D by reductive amination reaction are provided. The reaction between a compound of Formula V, VI, VII, VIII or IX or a compound of Table D and an aldehyde containing biomolecule, such as a glycan can be conducted under conditions that facilitate reductive amination in the presence of a reducing agent, such as from sodium cyanoborohydride or sodium triacetoxyborohydride. The reaction can be carried out in a solution or suspension of a compound of Formula V, VI, VII, VIII or IX or a compound of Table D in an acidic media, for example in citric acid or acetic acid, and mixing with a solution or suspension of a reducing agent such as cyanoborohydride or sodium triacetoxyborohydride in an organic solvent, for example, tetrohydrofuran or dimethylsulfoxide.

Methods are provided for analyzing a biomolecule containing an aldehyde group, such as a glycan, in a sample containing at least one biomolecule, such as a glycan, by means of liquid chromatography and mass spectrometry. The analytical method comprises the steps of labeling the biomolecule, such as a glycan, in the sample by reacting with a compound of Formula V, VI, VII, VIII or IX or a compound of Table D for a time and under conditions suitable to facilitate the labeling through reductive amination; and subjecting the labeled conjugate to liquid chromatography and mass spectrometry.

Analytical kits for assaying biomolecules, such as glycans, can include (i) a labeling module comprising a compound of Formula VI, VII, VIII, IX or a compound of Table D, and salts and solvate thereof; and optionally one or more of the following:
(i) a deglycosylation module comprising one or more endoglycosidases, a buffer, and one or more surfactants, or one or more compounds that can perform a chemical release of glycoprotein; and
(ii) a separation device for clean-up such as a solid phase extraction device or a centrifugal filtration device or the like.

Glycans conjugated to MS active fluorescent compounds of Formula V, VI, VII, VIII or IX, and salts or solvates thereof are also provided.

### Phenyl Based MS Active Fluorescence Tagging Compounds

In another embodiment, analytical kits for assaying glycans and other biomolecules include (i) a labeling module comprising a compound of Formula X, XA, XB, XC, XD, XE, XF or XG and salts and solvate thereof; and optionally one or more of the following is provided:
(i) a deglycosylation module comprising one or more endoglycosidases, a buffer, and one or more surfactants, or one or more compounds that can perform a chemical release of glycoprotein; and
(ii) a separation device for clean-up such as a solid phase extraction device or a centrifugal filtration device or the like.

Kits comprising MS active, fluorescence tagging compounds can contain a phenyl derivative of Formula X:

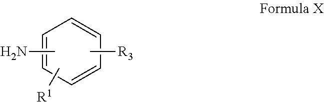

wherein R₁ is selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R³ is

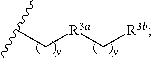

R^{3a} is selected from ester, amide, amine, oxygen, urea, carbamate, carbonate, sulfur, thiourea, thiocarbamate, alkyl or carbonyl;
R^{3b} is

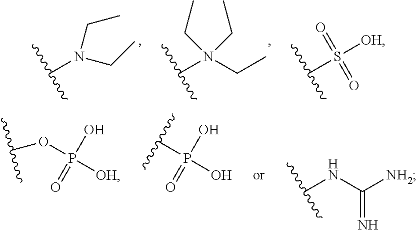

y=0−12;
z=1−12;
and salts or solvates thereof.

Kits comprising MS active, fluorescence tagging compounds can contain a phenyl derivative of Formula XA:

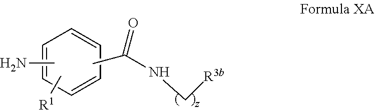

wherein R₁ is selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

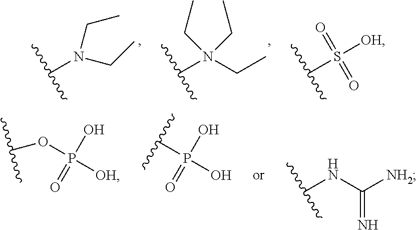

z=1−12;
and salts or solvates thereof.

Kits comprising MS active, fluorescence tagging compounds can contain a phenyl derivative of Formula XB:

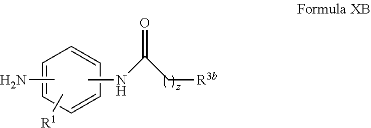

wherein R₁ is selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

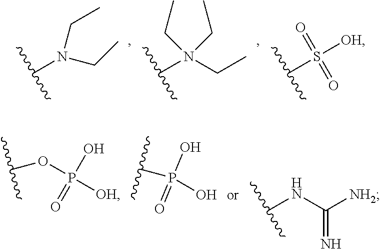

z=1−12;
and salts or solvates thereof.

Kits comprising MS active, fluorescence tagging compounds can contain a phenyl derivative of Formula XC:

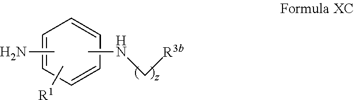

wherein R₁ is selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

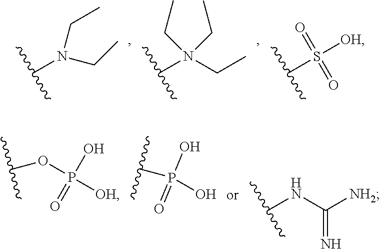

z=1−12;
and salts or solvates thereof.

Kits comprising MS active, fluorescence tagging compounds can contain a phenyl derivative of Formula XD:

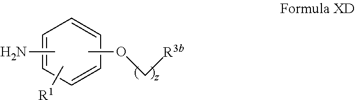

wherein R₁ is selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

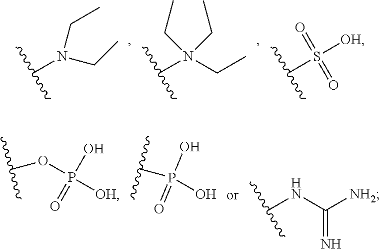

z=1−12;
and salts or solvates thereof.

Kits comprising MS active, fluorescence tagging compounds can contain a phenyl derivative of Formula XE:

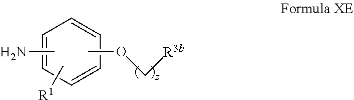

wherein R₁ is selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

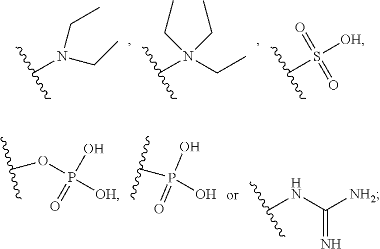

z=1−12;
and salts or solvates thereof.

Kits comprising MS active, fluorescence tagging compounds can contain a phenyl derivative of Formula XF:

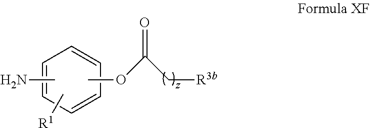

wherein R₁ is selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

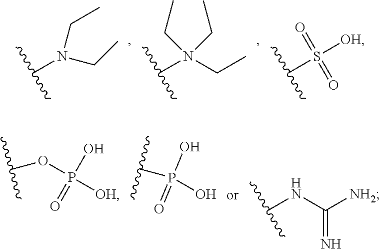

z=1−12;
and salts or solvates thereof.

Kits comprising MS active, fluorescence tagging compounds can contain a phenyl derivative of Formula XG:

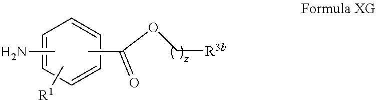

wherein R₁ is selected from hydrogen, halogen, lower alkyl, lower alkenyl, lower alkynyl, lower alkanoyl, lower haloalkyl, lower haloalkenyl, lower haloalkynyl, lower cycloalkyl, lower acyloxy, hydroxy, amino, lower alkylamino, amido, nitro, lower alkylthio, lower alkylsulfinyl, lower alkylsulfonyl, sulfonate, sulfonic acid, N₃, SH, SCH₃, C(O)CH₃, CO₂CH₃ and CO₂H;
R^{3b} is

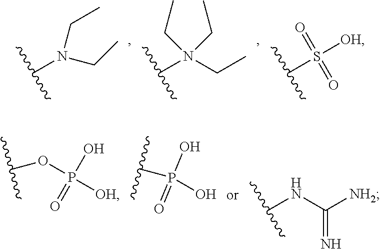

z=1−12;
and salts or solvates thereof.

In addition, provided below are kits containing exemplary compounds (Table E) of the structural Formulas X, XA, XB, XC, XD, XE, XF or XG which can be useful for fluorescent labeling of glycans and subsequent analysis by means of liquid chromatography and mass spectrometry. The compounds of structural formulas X, XA, XB, XC, XD, XE, XF or XG can be optionally substituted with any group that does not substantially reduce the fluorescence of the compound.

TABLE E

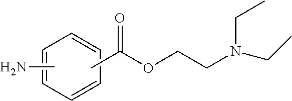

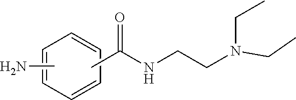

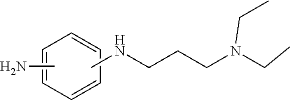

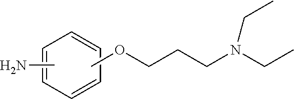

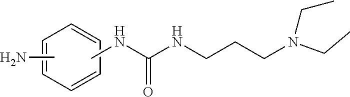

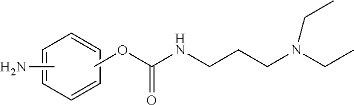

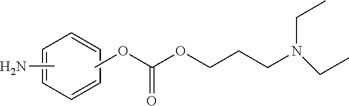

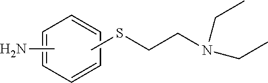

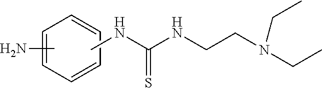

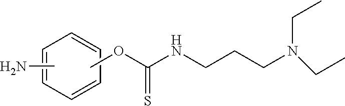

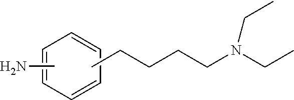

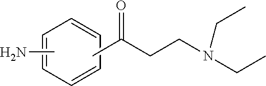

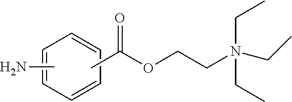

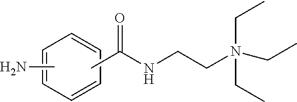

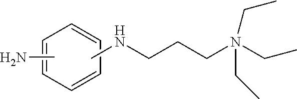

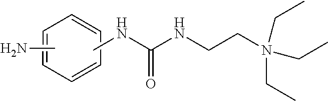

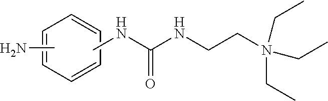

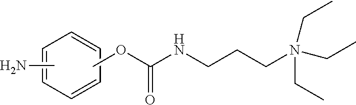

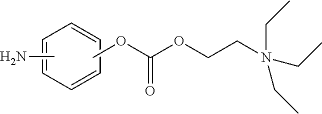

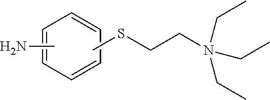

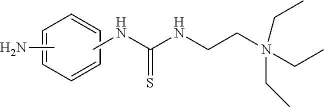

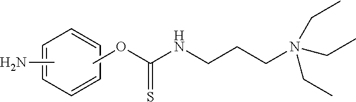

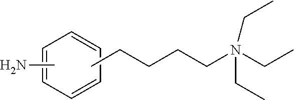

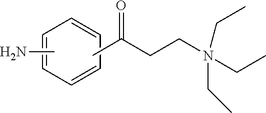

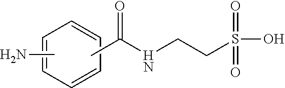

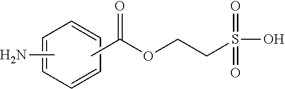

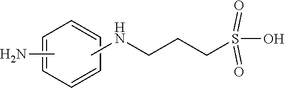

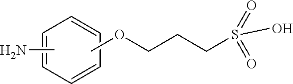

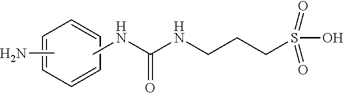

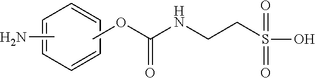

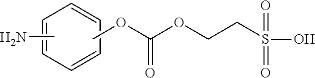

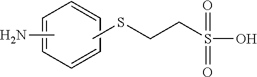

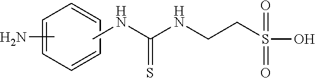

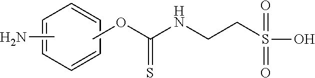

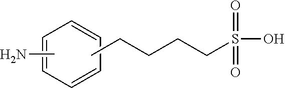

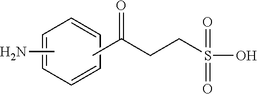

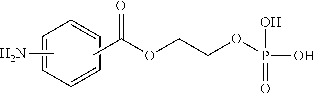

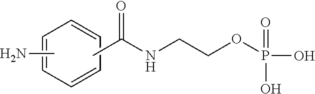

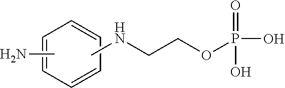

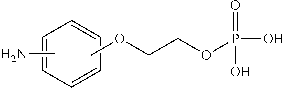

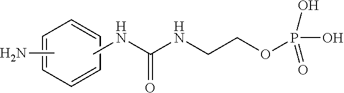

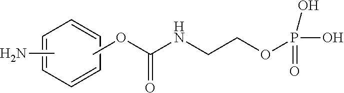

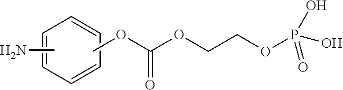

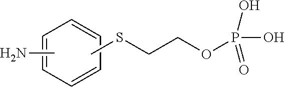

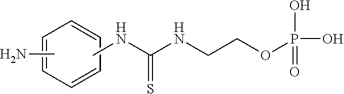

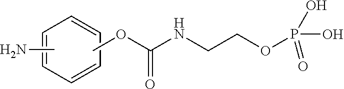

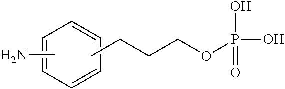

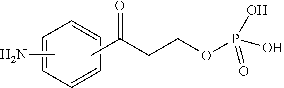

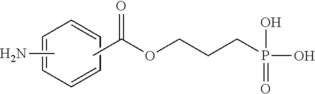

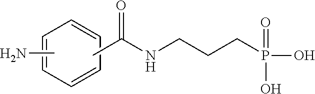

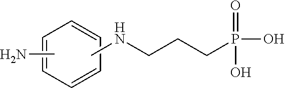

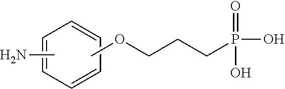

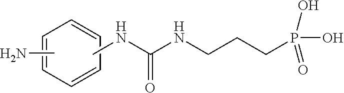

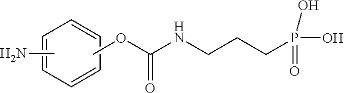

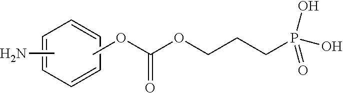

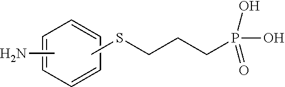

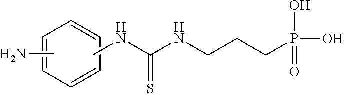

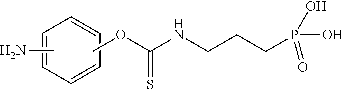

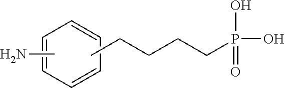

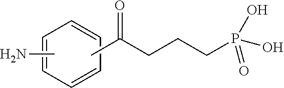

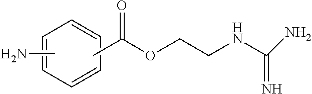

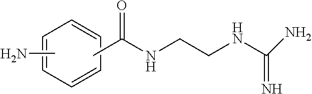

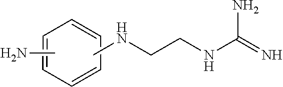

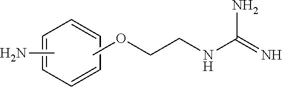

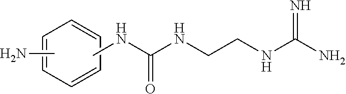

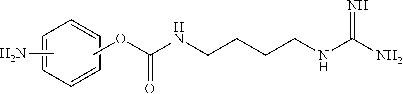

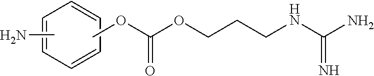

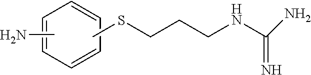

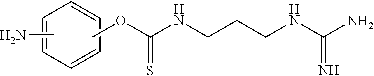

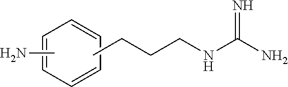

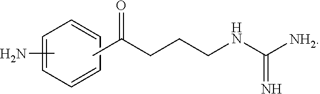

Methods for tagging, derivatizing or conjugating glycans and other biomolecules containing at least one ketone group or an aldehyde group with a compound of Formula XIV, XIVA, XIVB, XIVC, XIVD, XIVE, XIVF or XIVG by reductive amination reaction are provided. The reaction between the compound of Formula X and an aldehyde containing biomolecule, such as a glycan can be conducted under conditions that facilitate reductive amination in the presence of a reducing agent, such as sodium cyanoborohydride or sodium triacetoxyborohydride. The reaction can be carried out in a solution or suspension of a compound of Formula X, XA, XB, XC, XD, XE, XF or XG in an acidic media, for example in citric acid or acetic acid, and mixing with a solution or suspension of a reducing agent such as cyanoborohydride or sodium triacetoxyborohydride in an organic solvent, for example, tetrohydrofuran or dimethylsulfoxide.

Methods for analyzing glycans and other biomolecules containing an aldehyde group in a sample containing at least one biomolecule, such as a glycan, by means of liquid chromatography and mass spectrometry. These analytical methods include the step of labeling the biomolecule, such as a glycan, in the sample by reacting with a compound of Formula X, XA, XB, XC, XD, XE, XF or XG for a time and under conditions suitable to facilitate the labeling through reductive amination; and subjecting the labeled conjugate to liquid chromatography and mass spectrometry.

As provided herein, glycans can be conjugated to MS active fluorescent compounds of Formula X and salts or solvates thereof. The following schematic shows the tagging of a glycan using a compound of Formula X through reductive amination:

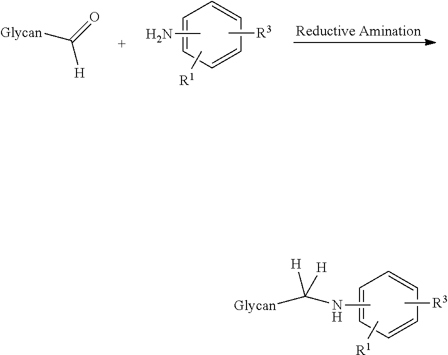

wherein FL R¹, R² and R³ are as described above.

Methods of labeling the glycans with MS active fluorescent compounds of Formula X, as well as conjugates resulting therefrom are provided.

The following Scheme I and Scheme II can be used to make the compounds described herein.

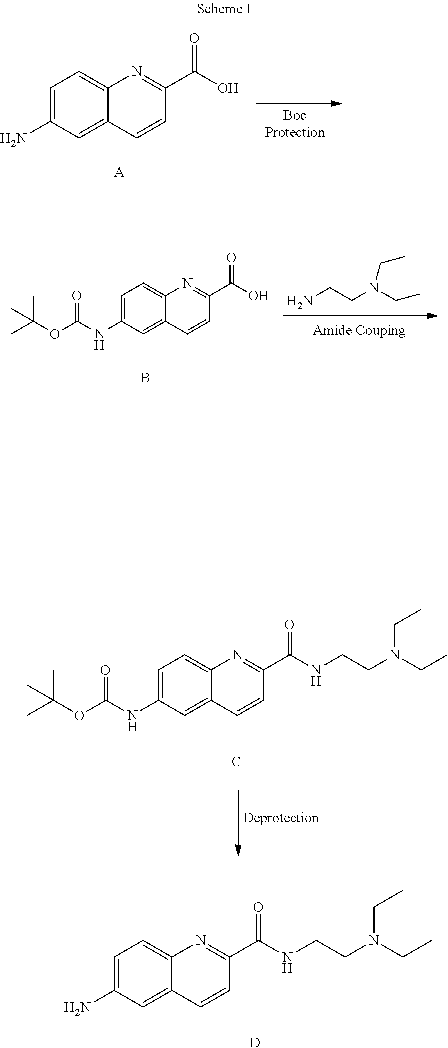

### Preparation of 6-amino-N-[2-(diethylamino)ethyl]-2-quinolinecarboxamide (D)

40 mg of B was dissolved n 2.5 mL of a 1:4 mixture of dimethylformamide:dichloromethane in a 10 mL flask equipped with a stir bar and purged with N₂. 1.7 mg of dimethylaminopyridine and 181 μL of dicyclohexylcarbodiimide were then added to the flask. After stirring for 10 min, 2-(diethylamino)ethylamine (57 mg) in 3 mL of dichloromethane was added to the flask. This was then stirred at room temperature for 20 hours. After this time, 3 mL of water was added to the reaction flask. The organic layer was separated and the aqueous layer was extracted with 2 mL of dichloromethane. The organic phases were combined, dried, and then evaporated to dryness to provide the crude material. This was subjected to standard organic chemistry purification techniques to provide the desired material C in >95% purity.

1.8 g of C was dissolved in a mixture of 5.3 g of trifluoracetic acid in 30 mL of dichloromethane. The reaction mixture was stirred at room temperature for 48 hours. After removal of the solvent under reduced pressure, the crude material was dissolved in 30 mL of 0.5 N HCl. This mixture was then extracted with 50 mL aliquots of ethyl acetate. The organic phases were combined, dried, and then evaporated to dryness to yield 1.2 gram of the crude product. This was subjected to standard organic chemistry purification techniques to provide the desired material D in >98% purity.

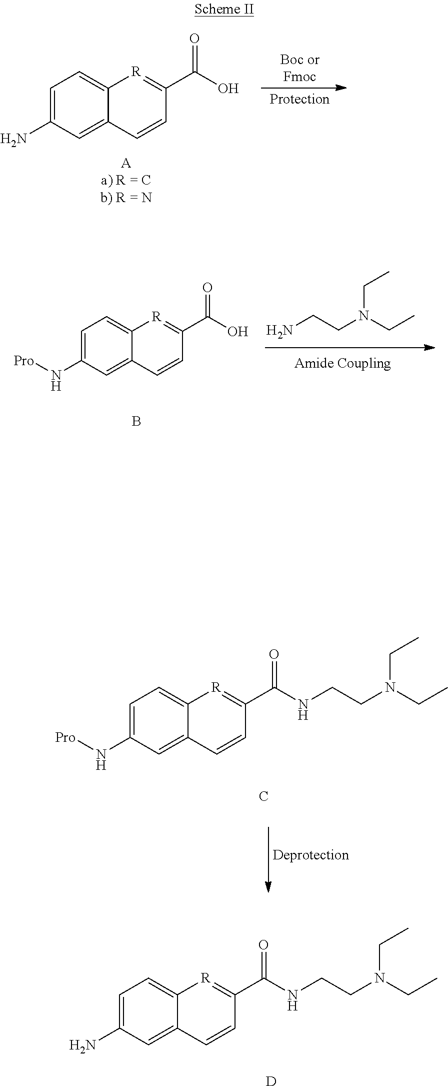

The method used for Scheme I is applicable to Scheme II for making the compounds presented herein.

### Other Derivatization Methods

A number of alternative derivatization procedures have been developed to permit the assay of glycans by high performance liquid chromatographic and electrophoretic separations. Under certain conditions, the compounds presented herein can be subject to rapid tagging processes where reagent solution is added to released glycans at room temperature for four or five minutes, and then lyophilized and subsequently reconstituted in acetonitrile/water solution. Other possible derivatization methods that might be utilized to tag glycans with the reagents described herein include:

(1) The o-phthalaldehyde (“OPA”)/mercaptan method. The OPA procedure can detect amino acids with a typical detectable level in the order of about 100 femtomole (fmol). Here, an adduct can be unstable and, therefore, should be prepared shortly before the detection step. Also, the reagent may not form a derivative with secondary amino acids.

(2) The 9-fluorenylmethylchloroformate (“FMOC”) method. The FMOC procedure provides for stable derivatives having a minimum detectable level in the order of a few hundred fmol. Free tryptophan and cystine are sometimes difficult to quantitate. The derivatizing reagent is preferably removed from the reaction mixture by an extraction step because it is itself fluorescent. The reagent has also been reported to form multiple derivatives with histidine. The reagent can be hazardous to work with, because it is corrosive and is a lachrymator.

(3) The phenylisothiocyanate (“PITC”) method. The PITC procedure yields stable derivatives which are formed rapidly. It can be used for both primary and secondary amino acids, as well as cystine. The method uses absorbance as the detection procedure, and can provide a minimum detection limit of 1 pmol. However, the derivatives are not fluorescent and detection must be performed at 254 nm, which does not allow for good detection selectivity.

(4) The dansyl chloride method. The dansyl chloride method provides stable derivatives with a minimum detectability in the order of about 1.5 pmol. It is able to detect secondary amines and cysteine, but it results in multiple derivatives.

(5) Fluorescent succinimidocarbamates are useful as derivatizing agents for amines, amino acids, peptides, phosphates and other classes of compounds. When the succinimidocarbamate reagent is used to tag a compound with a fluorescent group, a detection limit of about 1 pmol can be achieved. These reagents can be used in conjunction with modern separation techniques such as high performance liquid chromatography, thin layer chromatography or capillary electrophoresis.

### Detection of Derivatized Glycans by MS and Fluorescence

Most amino acids and/or glycans are not readily detectable in the absence of a strong chromophore or fluorophore or MS active moiety. The absorbance and fluorescence response are quite weak. One tactic used to maximize the sensitivity of an assay is to convert the compound of interest into a derivative that exhibits a better response for the detection method being utilized. The selection of a derivatizing agent is an important choice in the development of an analytical procedure. The derivatizing agent affects the ultimate sensitivity and accuracy of the analysis by maximizing the sensitivity, yield and stability of the derivatized molecules.

Basically, the following determinations can be performed separately: (1) the glycosylated sites; (2) the glycosylated site occupancy; (3) the structure and amount of each glycan at each site: and (4) the number of glycoforms. Harvey, D. J., Identification of Protein-Bound Carbohydrates by Mass Spectrometry, 1 PROTEOMICS 311-319 (2001) at 312, incorporated herein by reference. In most situations, MS can provide the answers to each of these steps. Hence the need for enhanced MS signals. Because of the branched nature of the glycan, however, structural determination of the glycan is complicated. Here, the monosaccharide unit, the anomericity and ring size of each monosaccharide, the monosaccharide sequence and ring conformation together with identification of other groups must be determined. With the exception of ring conformation, MS can be used directly or indirectly to make these determinations using MALDI and/or ESI as the preferred MS technique. Id. at 313-316, incorporated herein by reference.

Compounds described herein are useful for derivatizing glycans because they can form stable, highly fluorescent MS derivative compounds and conjugate glycans. The general methodology for an analysis of a glycan or amino acid derivatized by these compounds include three closely related processes: (1) formation of derivatives in the sample; (2) separation of the derivatives; and (3) detection of the separated derivatives. The first step is generally performed by reacting a mixture with one of the present compounds as a reagent to yield a derivatized compound. The derivatives provide a fluorescent signal which can then be detected in the detection stage of the analysis.

The separation step is based upon the differences in the chemical structure of the derivatives. The derivatized compounds can differ from each other in the same way that the chemical structures of the precursor compounds differ. The derivatives must be separated so that the detector signal can be correctly related to the concentration of each derivative. The derivatized glycans can be separated and detected by chromatography, e.g., by high performance liquid chromatography (“HPLC”) or capillary zone electrophoresis (“CZE”).

The detection step is generally carried out using either an absorbance or fluorescence detector. As each derivative is eluted from the chromatographic column after separation, its presence and quantity is detected by a mass spectrometer and/or by the aborbance or emission of light. The sensitivity of the assay depends upon the strength of the signal produced.

Analytical methods of analyzing glycans have become considerably sophisticated. Exemplary analytical instrumentation includes CE-, HPAEC-PAD, HILIC-LC/FLR, RPLC/MS, and MALDI-MS. Liquid chromatography (“LC”) separation with fluorescence detection is widely used in the pharmaceutical industry for the characterization of enzymatically/chemically released glycan, typically tagged with a fluorescent dye at the reducing end of a glycan. Kalyan R. Anumula & Shirish T. Dhume, High Resolution and High Sensitivity Methods for Oligosaccharide Mapping and Characterization by Normal Phase High Performance Liquid Chromatography Following Derivatization with Highly Fluorescent Anthranilic Acid, 8 GLYCOBIOLOGY 685 (1998); Karina Marino et al., A Systematic Approach to Protein Glycosylation Analysis: A Path Through the Maze, 6 NATURE CHEMICAL BIOLOGY 713 (2010).

Fluorescent measurements are sensitive and quantitative; the low detection limit is in the low femtomoles. With recent advancements in mass spectrometry instrumentation, the combination of liquid chromatography, fluorescence and MS has gained more popularity as an analytical instrument platform for routine characterization of fluorescently labeled N-linked glycans. Therefore, relative quantitation and molecular weight measurements can be done in a single analysis. Shigeo Suzuki et al., Comparison of the Sensitivities of Various Derivatives of Oligosaccharides in LC/MS with Fast Atom Bombardment and Electrospray Ionization Interfaces, 1006 ANAL CHEM 2073 (1996). However, a challenge has been that glycans do not ionize efficiently via electro-spray-ionization (“ESI”).

### Additional Uses for the Compounds Presented Herein

Absorbance detection is generally used in protein mapping work. Two different detection processes which are often used for this purpose are: a) detection at 210-215 nm using a single wavelength detector; and b) broadband spectral detection using a photodiode array (PDA) detector. In the first method, all peptides absorb at that wavelength, thus the user can ensure that all peptides eluted from the column are detected. One difficulty with this technique is that a wide variety of compounds absorb in this region of the spectrum, and extreme care must be taken to ensure that all reagents, eluents, glassware, etc. are scrupulously clean to ensure that the observed signal is solely from the peptides. In the second method, the PDA detector collects the spectra of the eluent at specific time intervals (e.g. a spectrum between 200 and 350 nm is collected every second). This provides more information than a single wavelength and thus can assist in distinguishing between peptides which can elute with similar retention times.

### Sample Preparation

To obtain high quality mass spectra, the condition of the sample is important. Compounds other than analyte will generally have an adverse effect on ion yield and are preferably removed. Indeed, while small amounts of sodium are essential for ionization by MALDI, carbohydrates are particularly susceptible to the effects of salts. Moreover, many carbohydrates occur as mixtures. Therefore, it is important to ensure that isolation and purification techniques do not cause fractionation of the sample with a loss of quantitative information.

We claim:

1. A method for conjugating a biomolecule containing at least one ketone group or aldehyde group, the method comprising:
reacting the biomolecule with a compound of Formula XB: 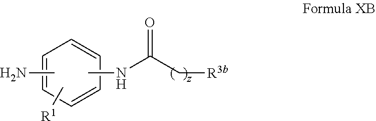
or a salt or solvate thereof, wherein:
R¹ is selected from the group consisting of hydrogen, halogen, C₁-C₆ alkyl, C₁-C₆ alkenyl, C₁-C₆ alkynyl, C₁-C₆ alkanoyl, C₁-C₆ haloalkyl, C₁-C₆ haloalkenyl, C₁-C₆ haloalkynyl, C₁-C₆ cycloalkyl, C₁-C₆ acyloxy, hydroxy, amino, C₁-C₆ alkylamino, amido, nitro, C₁-C₆ alkylthio, C₁-C₆ alkylsulfinyl, C₁-C₆ alkylsulfonyl, sulfonate, sulfonic acid, —N₃, —SH, —SCH₃, —C(O)CH₃, —CO₂CH₃ and —CO₂H;
R^{3b} is 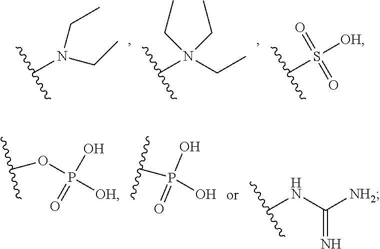
and
z=1−12;
wherein reacting the biomolecule with the compound of Formula XB is done under conditions that facilitate reductive amination in the presence of a reducing agent, thereby forming a conjugate.

2. The method of claim 1, wherein reacting the biomolecule comprises:
combining the biomolecule and the compound of Formula XB in an acidic media to form a mixture; and
mixing the mixture with a solution or suspension of the reducing agent in an organic solvent.

3. The method of claim 2, wherein the acidic media is a solution of citric acid or acetic acid.

4. The method of claim 1, wherein the reducing agent is sodium cyanoborohydride or sodium triacetoxyborohydride.

5. The method of claim 2, wherein the organic solvent is dimethylsulfoxide, tetrahydrofuran, dichloromethane, 1,2-dichloroethane, ethanol, methanol, isopropanol, toluene, xylene, or a mixture thereof.

6. The method of claim 1, wherein the biomolecule is a glycan.

7. The method of claim 1, wherein R₁ is hydrogen.

8. The method of claim 1, wherein z is 2 or 3.

9. The method of claim 1, wherein R^{3b} is

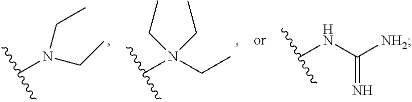

10. The method of claim 1, wherein the compound of Formula XB has the structure

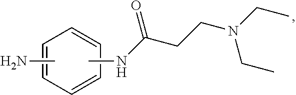

11. The method of claim 1, wherein R^{3b} is

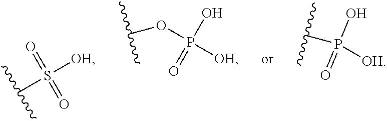 [SMILES: *S(=O)(=O)O ; *OP(=O)(O)O ; *P(=O)(O)O]

12. The method of claim 1, wherein the compound of Formula XB has a structure selected from the group consisting of:

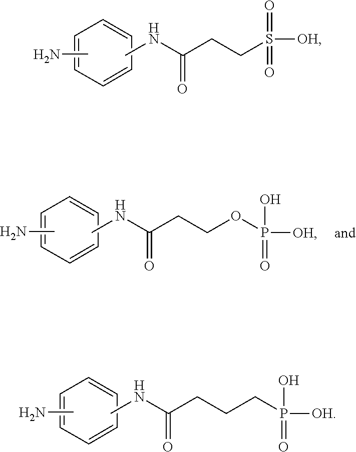

13. The method of claim 1, further comprising analyzing the conjugate with liquid chromatography and mass spectrometry.
